(19) United States
(12) Patent Application Publication
(10) Pub. No.: US 2004/0193086 A1
(43) Pub. Date: Sep. 30, 2004

(54) Dynamic position adjustment device for extremities of the human body

(76) Inventor: Ruth P. Cofre, Long Grove, IL (US)

Correspondence Address: Bell, Boyd & Lloyd LLC, P.O. Box 1135, Chicago, IL, 60690-1135, US

(21) Appl. No.: 10/413,658

(22) Filed: Apr. 15, 2003

Related U.S. Application Data
(63) Continuation-in-part of application No. 10/401,707, filed on Mar. 28, 2003.

(51) Int. Cl. A61F 5/00

(52) U.S. Cl. 602/20

(57) ABSTRACT

The present invention relates to a position adjustment device which includes one or more body securing members and one or more elongated members operatively coupled to the body securing members. The position adjustment device re-positions body portions from abnormal or mis-aligned positions to normal or more normal positions. The position adjustment device also provides users with dynamic support for use while engaging in activities, as well as posture enhancement and other health benefits.

### PRIORITY CLAIM

This application is a continuation-in-part patent application of U.S. patent application, Ser. No. 10/401,707, filed on Mar. 28, 2003, entitled “Dynamic Position Adjustment Device for Portions of the Human Body.”

### BACKGROUND OF THE INVENTION

Various medical conditions can cause joints, shoulders, backs, hips and other portions of the human body to become subluxated, dislocated, improperly aligned or otherwise abnormally positioned. These medical conditions can include, among others, loss of muscle strength, strokes, injuries, trauma, stress, nerve damage, diminished blood supply, abnormalities, birth defects and other musculoskeletal disorders.

There are known slings, braces and restraints which are adapted to provide support to afflicted portions of the body. These devices have several disadvantages. Many of these devices restrain limbs in a predetermined position. Such devices do not allow people to move their limbs through a relatively significant range of motion in daily activities. Other devices, such as the sling, allow the person to move his/her limb but fail to dynamically support the limb in motion. Such devices therefore do not provide sufficient assistance in the proper positioning of the limb when in motion.

One known hand brace includes a hand glove connected to a strap. This hand brace attempts to treat a hand which is abnormally inverted or pronated. The wearer inserts his/her hand into the glove and wraps the strap around his/her arm. The wearer ties the free end of the strap to his/her upper arm. This hand brace has been found to provide inadequate re-positioning of the abnormally pronated hand.

Therefore, there is a need to overcome each of these disadvantages to help people afflicted with one or more of the medical conditions described above. There is also a need to provide improved devices for such people which are relatively discrete, comfortable and convenient and which provide wearers with dynamic bodily support during daily activities to improve and enhance their quality of life.

### SUMMARY OF THE INVENTION

The present invention relates to position adjustment devices for portions of the human body. In particular, the present invention relates to different embodiments of a position adjustment device which: (a) position shoulders in a normal or more normal position in relation to the upper body; (b) compress abnormally protruding scapulas; (c) reduce subluxation or dislocation of shoulders from the shoulder sockets; (d) decrease load on shoulder tissue, and (e) provide abnormal and lower back support.

In one embodiment of present invention, the position adjustment device includes a single piece that re-positions a shoulder which abnormally extend forward to a normal or more normal position. Part of the device wraps around a portion of the upper arm and shoulder to form a sleeve and is secured in place by interconnecting flaps. Another part of the device is wrapped around the side of the upper body and is secured to the upper body by straps that wrap around the waist of the user and attach to one another. The shoulder is re-positioned by a number of straps that connect the arm and shoulder to the upper body. One strap is connected to the top of the shoulder and is wrapped around the upper body to primarily compress the shoulder into the shoulder socket. Another strap, connected to the front of the shoulder and directed over the shoulder and across the back, attaches at the waist to pull the shoulder backward. This strap forces the scapula against a relatively rigid piece of plastic. This piece of plastic is located in the back part of the device to compress and hold the scapula in place. Another strap is connected to the back of the arm and attaches to the middle of the back to further rotate and pull the shoulder backward.

In another embodiment of the present invention, the position adjustment device includes a device made up of a single piece that repositions a shoulder which abnormally extend forward to a normal or more normal position, and which also re-distributes the weight of the arm to the torso. Part of the device wraps around a portion of the upper arm and shoulder to form a sleeve and is secured in place by interconnecting flaps. Another part of the device is wrapped around the side of the upper body and is secured to the upper body by straps that wrap around the waist of the user and attach to one another. The shoulder is re-positioned by a number of straps that connect the arm and shoulder to the upper body. One strap is connected to the top of the shoulder and is wrapped around the upper body to primarily compress the shoulder into the shoulder socket. Another strap is connected to the back of the arm and attaches to the middle of the back to rotate and pull the shoulder backward. A pair of straps is connected to the end of the sleeve and cross one another at the top of the shoulder where a fastener attaches them to the shoulder area. One of the pair of straps crosses the front of the user and the other crosses the back of the user to attach at the waist on the opposite side. These straps reduce the load on the shoulder tissue and also pull the shoulder into the shoulder socket to reduce subluxation during movement.

Another embodiment of the present invention includes a position adjustment device made up of a single unit that re-positions both shoulders which abnormally extend forward to a normal or more normal position. The device also re-positions portions of the upper body to enhance breathing. The device wraps around the front of the upper body including the shoulders, and attaches to itself in the back. A strap is connected to the front of both shoulders. These straps pull the shoulders back by wrapping around the sides of the shoulders and attaching to the back portion of the device.

Another embodiment of the present invention includes a position adjustment device including an upper unit and a lower unit which re-position both shoulders which abnormally extend forward, and which also provide abdominal and lower back support. The upper unit wraps around the front of the upper body including the shoulders, and attaches to itself in the back. A strap is connected to the front of both shoulders. These straps pull the shoulders back by wrapping around the sides of the shoulders. These straps are preferably directed slightly downward as they cross one another in the back. The lower unit wraps around the lower torso area. Two relatively long straps cross the front of the weaver, and extend over the shoulders and connect to the back waist area of the wearer. The straps pull the shoulders backward and also provide an elastic or spring force to the upper body. This spring force provides abdominal support and support to the lower back.

Another embodiment of the present invention includes an extremity position adjustment device which is adapted for use in conjunction with either one of the position adjustment devices described above. The extremity position adjustment device, in one embodiment, includes a hand securing member and a detachable strap. One end of the strap is removably attached to the hand securing member, and the other end of the strap is removably attached to a portion of one of the position adjustment devices. The position adjustment device serves the role as an anchor for the strap. This extremity position adjustment device dynamically re-positions and supports abnormally positioned extremities, such as hands.

It is therefore an advantage of the present invention to provide a position adjustment device for portions of the human body. Yet another advantage of the present invention is to decrease load on tissues of abnormally positioned body portions. Another advantage of the present invention is to re-position abnormally positioned shoulders during while the shoulders are at rest or in motion.

Still another advantage of the present invention is to aid in the biomechanical alignment of the shoulder girdle by re-positioning the humeral head relative to the glenoid fossa.

Another advantage of the present invention is to provide undergarment dynamic bodily support and bodily position adjustment in a manner which is relatively discrete. Yet another advantage of the present invention is to reduce or prevent pain caused by misalignment or dislocation of body portions.

Another advantage of the present invention is to reduce or prevent subluxation.

Still another advantage of the present invention is to protect the shoulder girdle during functional activities by decreasing overstretching of the serratus anterior muscle and other muscles and connective tissue associated with the shoulder girdle.

Another advantage of the present invention is to provide therapeutic intervention and rehabilitation by helping to maintain joint and tissue integrity to body portions requiring acute or chronic re-positioning.

Yet another advantage of the present invention is to aid in stabilizing the scapula against the thoracic wall to improve active shoulder control and strength.

Still another advantage of the present invention is to aid in the proper biomechanical alignment of the trunk by providing abdominal support and lumbar thoracic stabilization.

Another advantage of the present invention is to prevent shoulders from rounding and elevating arms.

Yet another advantage of the present invention is to improve posture through a broad range of movement.

Still another advantage of the present invention is to dynamically re-position abnormally positioned extremities.

Additional features and advantages of the present invention are described in, and will be apparent from, the following Detailed Description of the Invention and the figures.

### BRIEF DESCRIPTION OF THE FIGURES

FIG. 1 is a front elevation view of the position adjustment device installed on the left side of the subject in one embodiment of the present invention.

FIG. 2 is a rear elevation view of the position adjustment device installed on the left side of the subject in one embodiment of the present invention.

FIG. 3 is a rear elevation view of the position adjustment device installed on the left side of the subject illustrating the loosening of a re-positioning member in one embodiment of the present invention.

FIG. 4 is an open top view of the external surface of the position adjustment device for the left side of a subject in one embodiment of the present invention.

FIG. 5 is an open top view of the internal surface of the position adjustment device for the left side of a subject in one embodiment of the present invention.

FIG. 6 is a front elevation view of a rear engagement member of the position adjustment device in one embodiment of the present invention.

FIG. 7 is a cross-sectional view of the rear engagement member of the position adjustment device of FIG. 6, taken substantially along line 7-7.

FIG. 8 is a top or plan view of the forces exerted by the position adjustment device on the subject in one embodiment of the present invention.

FIG. 9 is a front elevation view of the position adjustment device installed on the left side of a subject in one embodiment of the present invention.

FIG. 10 is a rear elevation view of the position adjustment device installed on the left side of a subject in one embodiment of the present invention.

FIG. 11 is a front elevation view of the position adjustment device installed on the left side of a subject illustrating the loosening of re-positioning members in one embodiment of the present invention.

FIG. 12 is a rear elevation view of the position adjustment device installed on the left side of a subject illustrating the loosening of re-positioning members in one embodiment of the present invention.

FIG. 13 is an open top view of the external surface of the position adjustment device for the left side of a subject in one embodiment of the present invention.

FIG. 14 is an open top view of the internal surface of the position adjustment device for the left side of a subject in one embodiment of the present invention.

FIG. 15 is a front elevation view of the position adjustment device installed on a subject in one embodiment of the present invention.

FIG. 16 is a rear elevation view of the position adjustment device installed on a subject in one embodiment of the present invention.

FIG. 17 is an open top view of the external surface of the position adjustment device in one embodiment of the present invention.

FIG. 18 is an open top view of the internal surface of the position adjustment device in one embodiment of the present invention.

FIG. 19 is a cross-sectional view of the rear portion of the position adjustment device of FIG. 17, taken substantially along line 19-19.

FIG. 20 is a front elevation view of the position adjustment device installed on a subject in one embodiment of the present invention.

FIG. 21 is a rear elevation view of the position adjustment device installed on a subject in one embodiment of the present invention

FIG. 22 is an open top view of the external surface of an upper portion of the position adjustment device in one embodiment of the present invention.

FIG. 23 is an open top view of the internal surface of an upper portion of the position adjustment device in one embodiment of the present invention.

FIG. 24 is a front perspective view of a lower portion of the position adjustment device in one embodiment of the present invention.

FIG. 25 is a rear perspective view of the position adjustment device illustrating the opening of a portion of the position adjustment device in one embodiment of the present invention.

FIG. 26 is a cross-sectional view of the position adjustment device of FIG. 20, taken substantially along line 26-26.

FIG. 27 is a front elevation view of the extremity position adjustment device used with a position adjustment device in one embodiment of the present invention.

FIG. 28 is a rear elevation view of the extremity position adjustment device used with a position adjustment device in one embodiment of the present invention.

FIG. 29 is a top or plan view of the palm side or front side of the extremity securing member in one embodiment of the present invention.

FIG. 30 is a top or plan view of the back hand side or back side of the extremity securing member in one embodiment of the present invention.

FIG. 31 is a top or plan view of the exterior surface of the extremity securing member shown in an open configuration in one embodiment of the present invention.

FIG. 32 is a top or plan view of the interior surface of the extremity securing member shown in an open configuration in one embodiment of the present invention.

FIG. 33 is a top or plan view of the exterior surface and top side of the extremity re-positioning member in one embodiment of the present invention.

FIG. 34 is a top or plan view of the interior surface and bottom side of the extremity re-positioning member in one embodiment of the present invention

### DETAILED DESCRIPTION OF THE INVENTION

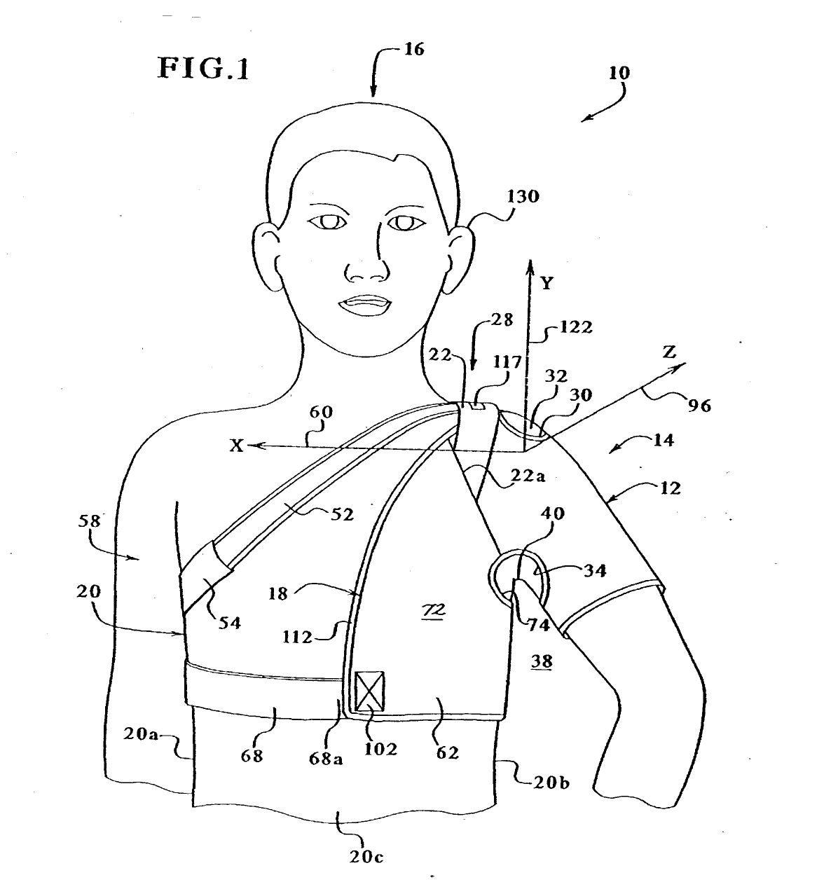

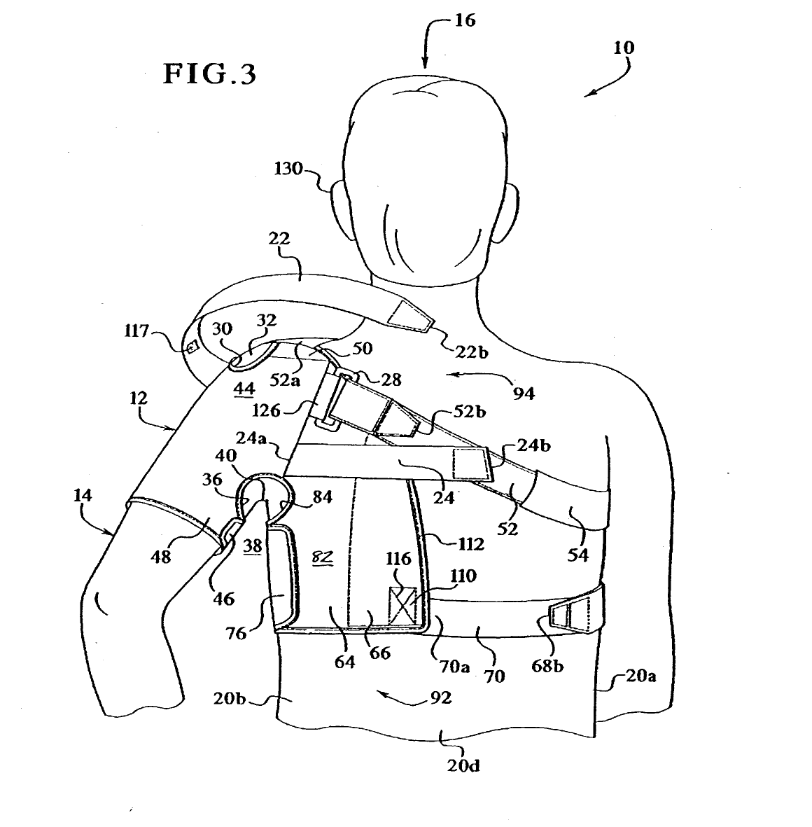

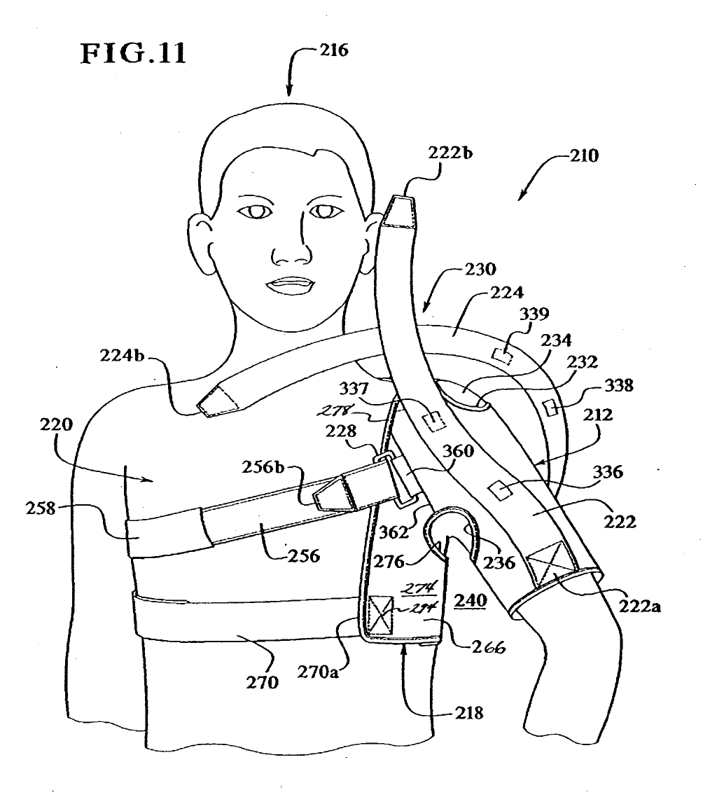

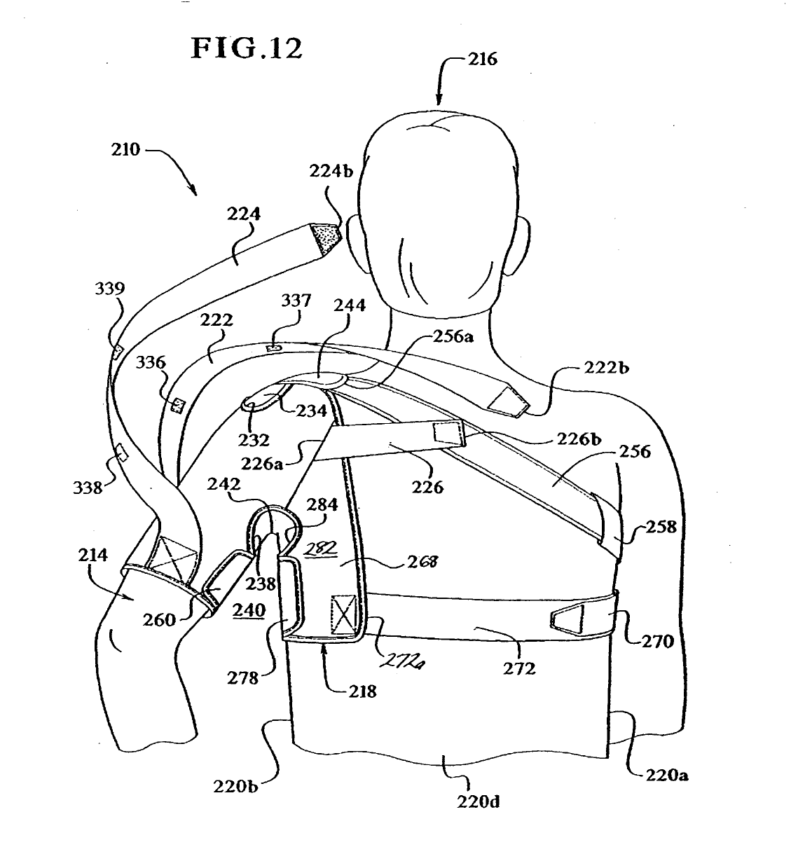

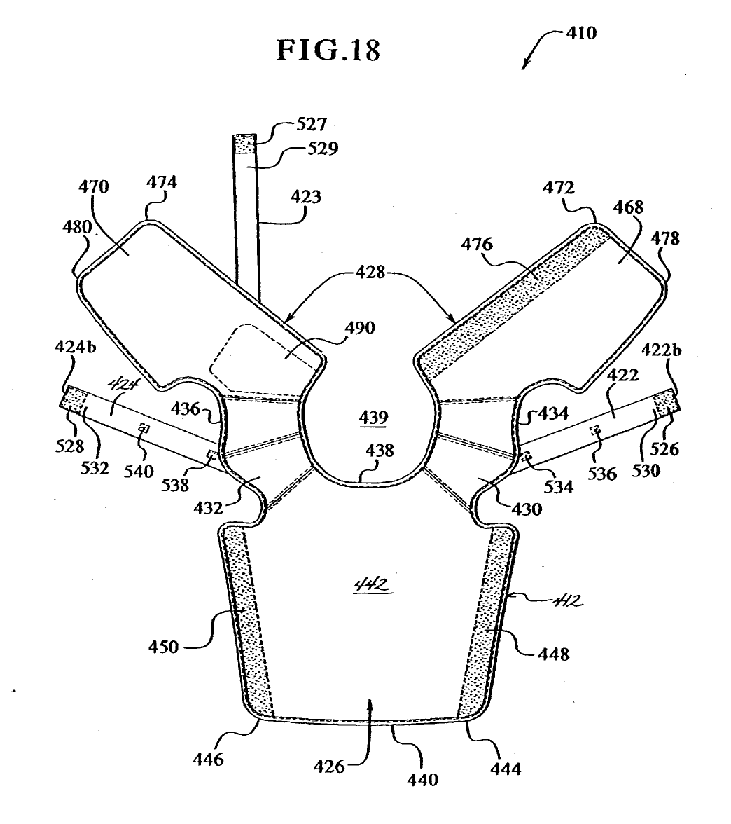

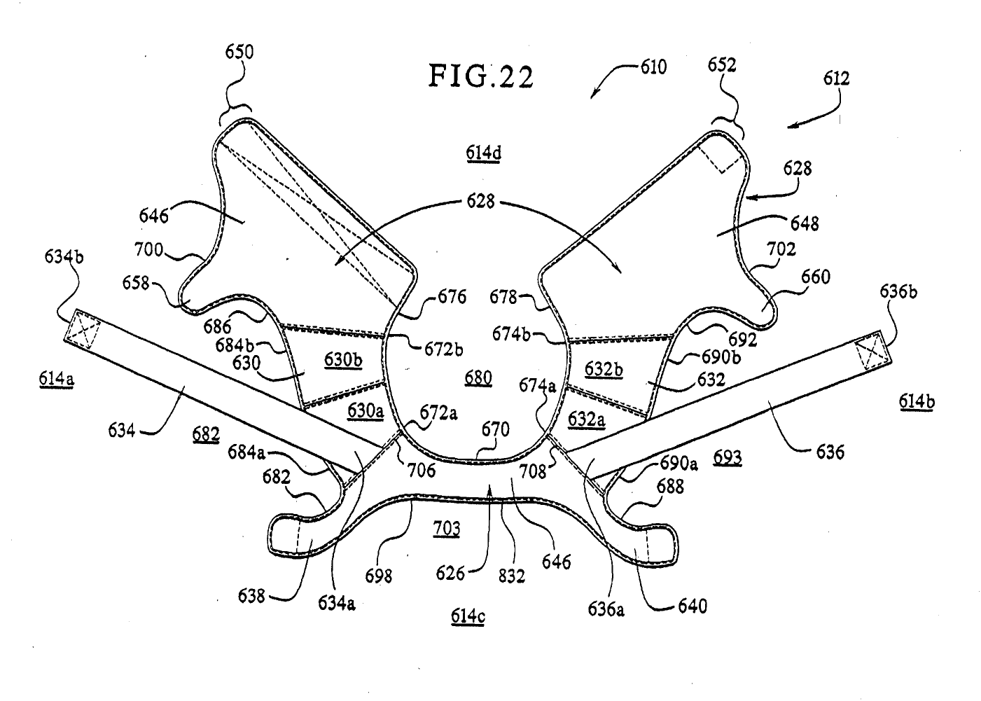

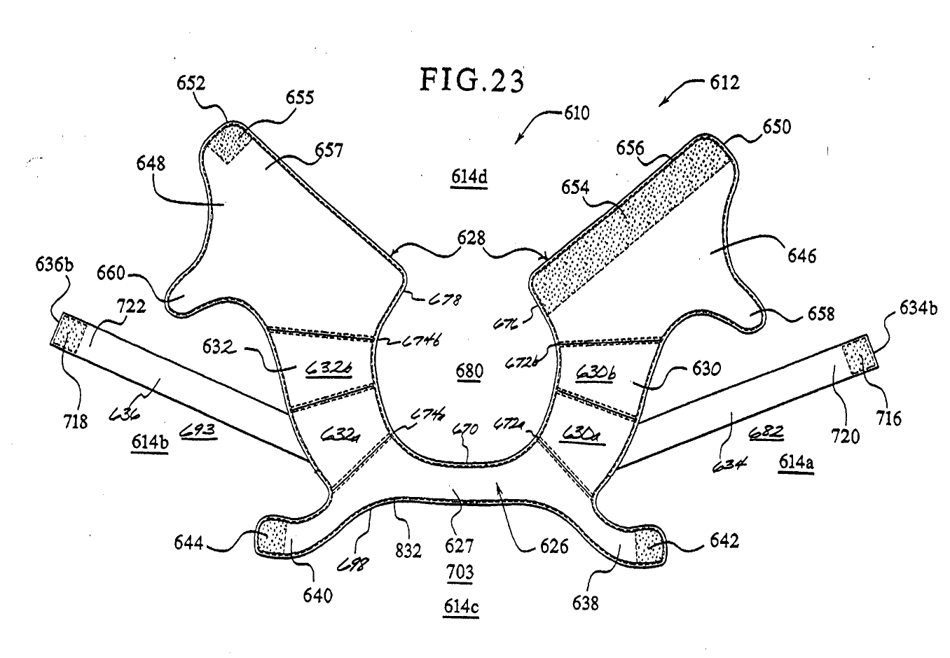

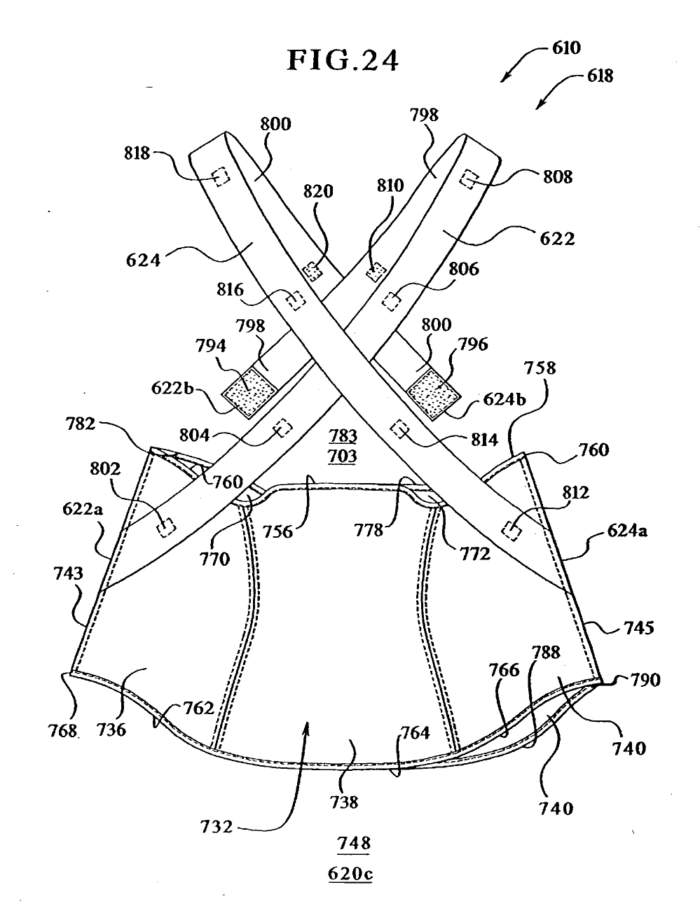

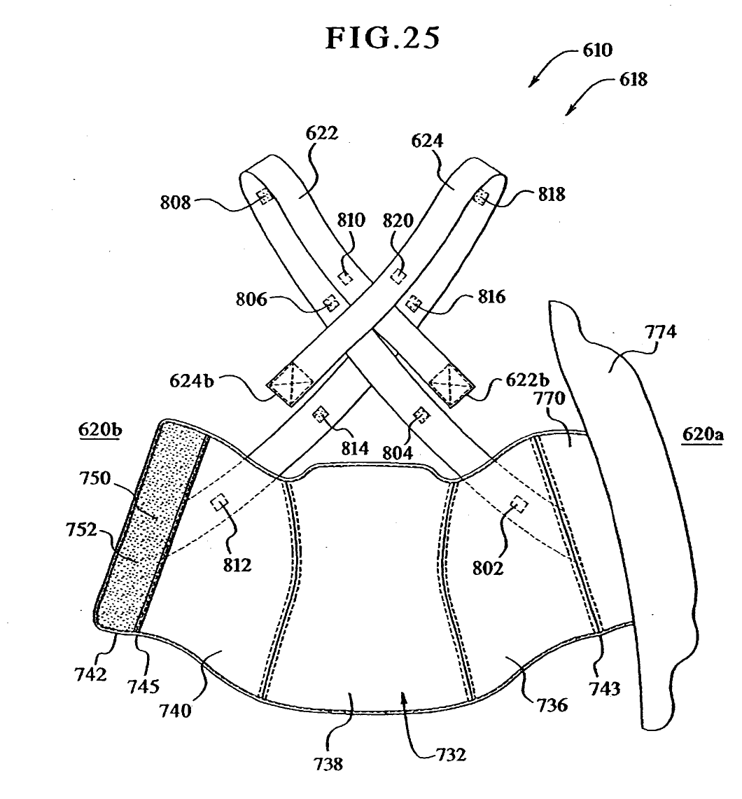

Referring now to the drawings, FIGS. 1 to 26 illustrate different embodiments of the position adjustment device of the present invention. As described below, depending upon the particular embodiment, the position adjustment device of the present invention can treat a person's shoulders, scapula, abdominal muscles, lower back and other body portions. In addition, the position adjustment device of the present invention can adjust a person's posture.

### I. First Embodiment

As illustrated in FIGS. 1 to 7, in one embodiment the position adjustment device 10 includes: (a) an arm securing member 12 which is secured to an abnormally positioned upper arm 14 or other body portion of a subject 16; (b) a torso securing member 18 which is engaged with the subject's upper torso 20 or other body portion which preferably serves as an anchor; (c) an elongated re-positioning member 22 which connects the securing member 12 to the torso securing member 18; (d) an elongated re-positioning member 24 which connects one portion of the arm securing member 12 to another portion of the arm securing member 12 as described below; and (e) one or more engagement members 26 connected to both the arm securing member 12 and the torso securing member 18.

### A. Arm Securing Member

The arm securing member 12 is preferably an arm holder or sleeve member which is configured to have a tubular shape fitting around the upper arm 14 and shoulder 28 area of the subject 16. It is also preferable that the arm securing member 12 includes a shoulder wall 30 which defines an adjustment slot, flex slot or shoulder opening 32. The shoulder wall 30 receives the protruding, substantially round shoulder 28. Accordingly, the shoulder wall 30 facilitates the secure attachment of the arm securing member 12 to the shoulder 28 when the subject 16 moves his/her shoulder to various positions.

In addition, the arm securing member 12 preferably includes a plurality of under arm walls 34 and 36 which contribute to defining an armpit cavity or under arm cavity 38. The under arm cavity 38 allows the under arm surface 40 of the subject 16 to receive ventilation. The under arm cavity 38 also enhances the comfort of the subject 16 and facilitates the free movement of the subject's arm 14 when the subject 16 is wearing the position adjustment device 10.

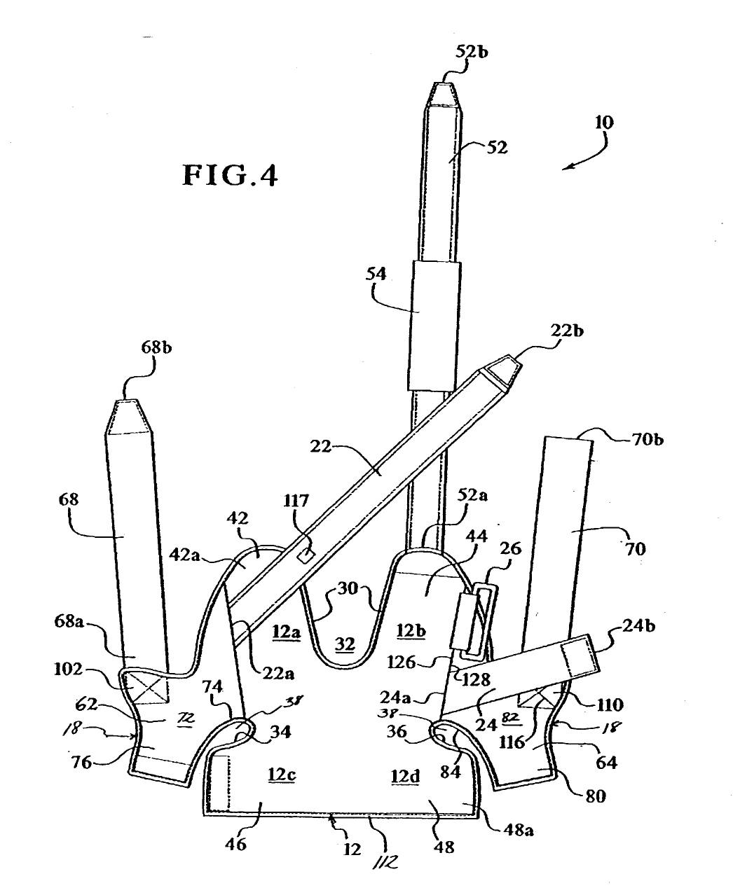

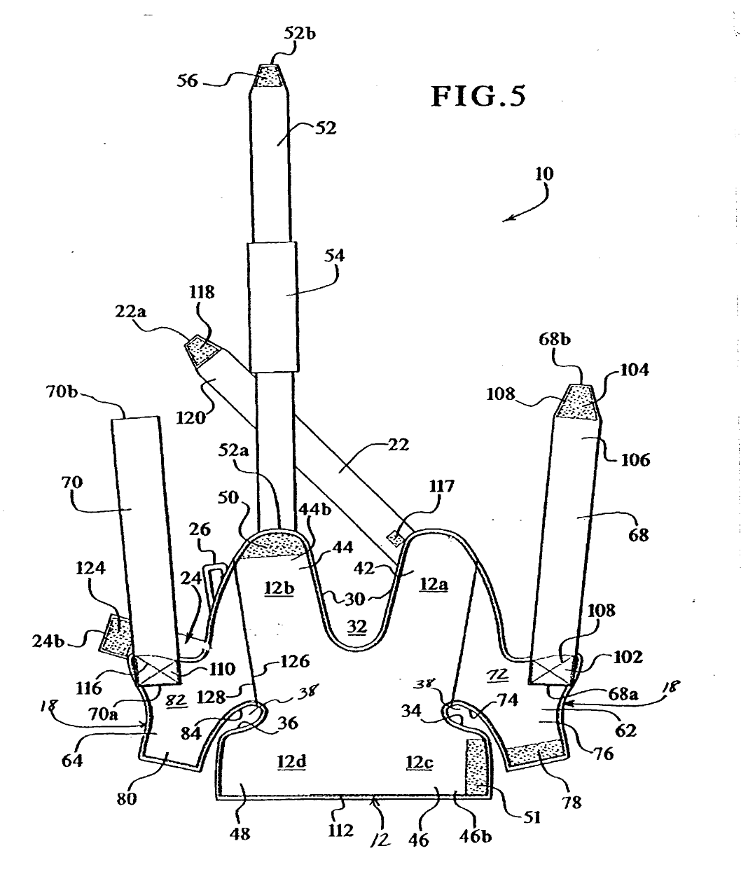

As best shown in FIGS. 4 and 5, the arm securing member 12 preferably has four arm members 42, 44, 46 and 48. Arm member 42 is located in the upper (or superior) and front (or anterior) portion 12a of the arm securing member 12. Arm member 44 is located in the upper (or superior) and rear (or posterior) portion 12b of the arm securing member 12. Arm member 46 is located in the lower (or inferior) and front (or anterior) portion 12c of the arm securing member 12. Arm member 48 is located in the lower (or inferior) and rear (or posterior) portion 12d of arm securing member 12. It is preferable that arm member 44 includes a re-adjustable fastener 50 such as a hook-type fastener, attached on an underside 44b of the arm member 44. When installed on the subject 16, arm member 44 preferably intersects with and removably attaches to the outer or exterior side 42a of the arm member 42. In this configuration, arm members 42 and 44 encompass the upper area of the shoulder 28 of the subject 16. Similarly, a re-adjustable fastener 51 on an underside 46b of the arm member 46 attaches the lower portion 12c of the arm securing member 12 to an outer or exterior side 48a of the arm member 48. In this configuration, arm members 46 and 48 encompass the upper arm 14 of the subject 16.

Furthermore, the arm member 44 preferably includes an extension member 52 which is preferably a strap or belt with relatively reduced elasticity. Extension member 52 includes (a) ends 52a and 52b; (b) a cushioning or comfort member 54 positioned between ends 52a and 52b; and (c) a re-adjustable fastener 56, preferably of the hook-type, positioned at the end 52b. The end 52a is non-removably attached to the upper portion of arm member 44. When installed, the extension member 52 extends across the upper torso 20 of the subject 16, and the comfort member 54 is positioned under an arm 58 opposite the arm 14 of the subject 16. The end 52b removably engages the engagement member 26 as described below. By adjusting the engagement of the extension member 52 to the engagement member 26, different degrees of pressure or lines of force 60 can be applied in order to compress: (a) the shoulder 28 into a normal or more normal position; and (b) the subject's scapula (not shown) into a normal or more normal position. Accordingly, the arm member 44 can function as a shoulder compression member and a scapula compression member, causing the shoulder 28 and the scapula to be compressed into the body of the subject 16.

### B. Torso Securing Member

The torso securing member 18, which is coupled to the arm securing member 12, preferably includes a structure that is configured to engage at least a portion of the upper torso 20 of subject 16. It is preferable that the torso securing member 18 includes: (a) a front (or anterior) torso engagement member 62; (b) a rear or back side (or posterior) torso engagement member 64; (c) at least one stiffening member 66 connected to, and preferably housed within, the rear torso engagement member 64; and (d) one or more straps, extension members or engagement members 68 and 70 connected to torso engagement members 62 and 64, respectively.

It is preferable that the torso engagement members 62 and 64 are sewn or otherwise secured to the arm members 42 and 44, respectively, in a non-removable fashion. The front torso engagement member 62, connected to arm member 42, includes: (a) a relatively flat chest or abdomen engagement member 72; (b) an under arm wall 74 further defining the under arm cavity 38; (c) an extension or flap 76 of the front torso engagement member which removably fastens to the rear torso engagement member 64; and (d) a re-adjustable fastener 78 for connecting the engagement member 62 to the rear torso engagement member 64 described below. The readjustable fastener 78 is positioned along the front torso engagement member flap 76 of the front torso engagement member 62. The readjustable fastener 78 allows the front torso engagement member 62 to removably attach to a rear torso engagement member extension or flap 80 of the rear torso engagement member 64 under the arm 14 of the subject 16. The under arm wall 74, which further defines under arm cavity 38, reduces restraints on movement of the arm 14 and also enhances the comfort and ventilation of the subject 16.

The rear torso engagement member 64, connected to arm member 44, preferably includes: (a) a relatively flat back side (or posterior) torso engagement member 82 which is secured to the back side 20d of the subject 16; (b) an under arm wall 84 further defining the under arm cavity 38; (c) the stiffening member 66; and (d) an extension or flap 80 for connecting the rear torso engagement member 64 to the front torso engagement member 62.)

As best shown in FIG. 7, it is preferable that a reinforcement member 112 be attached to the edges of the securing members 12 and 18 to protect the layers of material of the position adjustment device 10 from separating, tearing, fraying or otherwise being damaged, and to aid in reinforcing and maintaining the shape of the brace. The reinforcement member 112 is preferably a binding or trim made of a flexible fabric or material having a relatively narrow uniform width sufficient to engage a portion of both outer and inner layers 86 and 88 of the securing members 12 and 18. The reinforcement member 112 engages the surface of the outer layer 86 of the securing members 12 and 18 and is wrapped over the edges of the outer and inner layers 86 and 88 to engage the surface of the inner layer 88. The reinforcement member 112 is preferably non-removably secured to the surfaces of the outer and inner layers 86 and 88 by a plurality of stitches 114. The reinforcement member 112 preferably has a length sufficient to form a continuous edge along the contours of the securing members 12 and 18. It should be appreciated that other devices and techniques can be used to reinforce the attachment of the inner layer 88 to the outer layer 86 of the securing members 12 and 18.

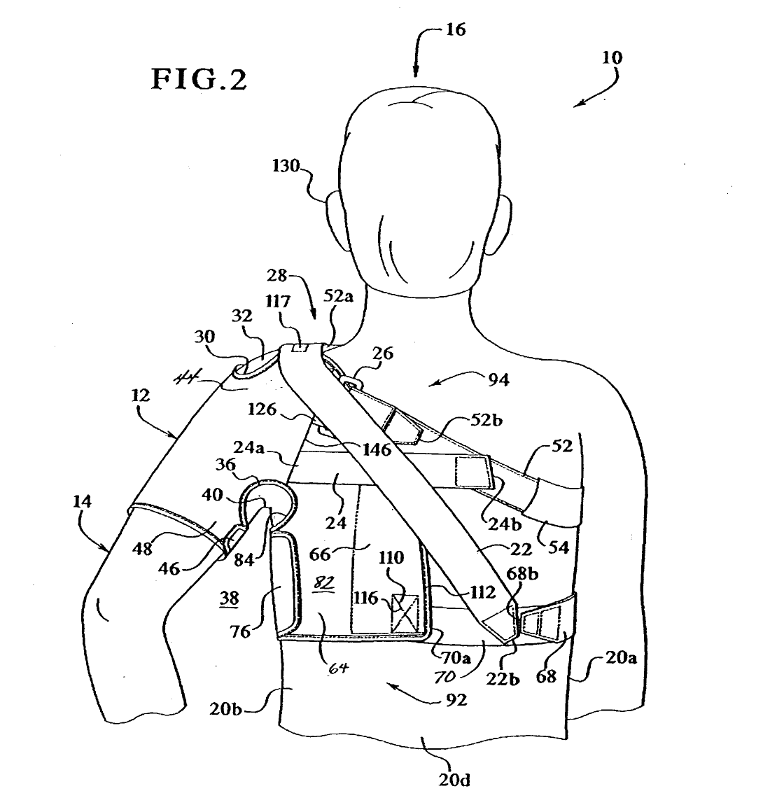

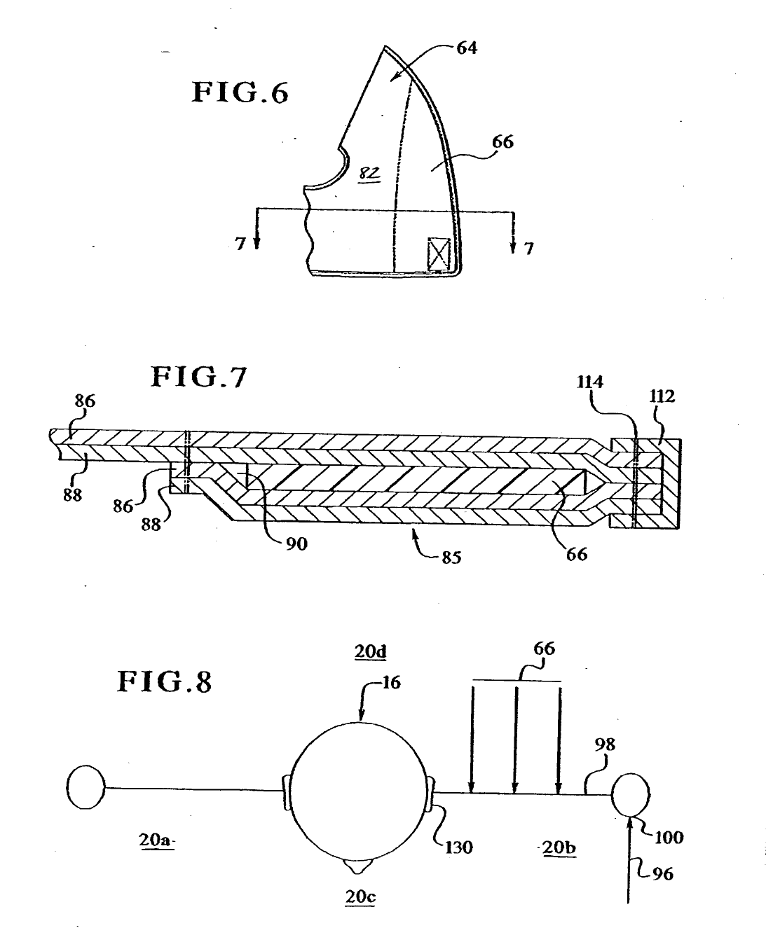

As best shown in FIGS. 6 and 7, the rear torso engagement member 64 preferably includes an additional panel 85 non-removably secured preferably by a plurality of stitches 87 to a portion of the inner layer 88 of the rear torso engagement member 64. The panel 85 is oriented such that the outer layer 86 of the panel is against the inner layer 88 of the rear torso engagement member 64 defining a space 90 in which the stiffening member 66 is contained. The orientation of panel 85 also allows the inner layer 88 of the panel 85 and the inner layer 88 of the rear torso engagement member 64 to form a common surface of the inner layer 88 to be placed against the skin of the subject 16. The stiffening member 66 preferably has a relatively flat shape and is positioned and sized to substantially fill the space 90. In addition, the stiffening member 66 extends from a lower area 92 of the subject's back 20d to an upper area 94 of the subject's back 20d. The stiffening member 66 functions, in part, as a scapula engagement member. The rigidity of the stiffening member 66 assists in stabilizing the scapula against the thoracic wall (not shown) of the subject 16. In addition, the stiffening member 66 can provide a fulcrum, pivot point or support point against which the re-positioning members 22 and 24 can each apply a re-positioning line of force 96 as discussed below. As illustrated in FIGS. 2 and 8, the stiffening force provided by the stiffening member 66 acts on a back location 98 of the subject 16 (preferably over the scapula), while the re-positioning line of force 96 acts on a front location 100 of the subject 16. Since the back location 98 is laterally offset from the front location 100, the stiffening member 66 enables a lever action. This lever action facilitates the re-positioning of the shoulder 28 which may be abnormally thrusted forward. This lever action also facilitates the re-positioning of the scapula which is abnormally rearwardly protruding. The stiffening member 66 can be constructed of any suitable material. It is preferable that the stiffening member 66 is constructed of a relatively light-weight plastic or polymer.

The torso securing members 62 and 64 of the securing member 18 preferably include adjustable straps or elongated securing members 68 and 70, respectively, which secure the torso engagement members 62 and 64 to the torso 20 of the subject 16. Securing member 68 has two ends 68a and 68b. The end 68a is attached in a non-removable fashion to a lower portion 102 of the front torso engagement member 62 and is oriented horizontally across the torso 20 of the subject 16. The free end 68b of securing member 68 includes a re-adjustable fastener 104. The fastener 104 is preferably secured to the underside 106 of end 68b using a plurality of stitches 108.

Similarly, securing member 70 has two ends 70a and 70b. The end 70a is non-removably attached to a lower portion 110 of the torso engagement member 64 and is oriented substantially horizontally across the torso 20 of the subject 16. The end 70b is positioned to receive the end 68b of the securing member 68. Securing members 68 and 70 connect the front and back torso engagement members 62 and 64 to the side 20a of the torso 20 and are preferably configured to wind or wrap around both sides 20c and 20d of the torso 20. Specifically, securing member 68 preferably wraps around the front side 20c of torso 20, and securing member 70 preferably wraps around the back side 20d of the torso 20. Securing member 68 overlaps and removably attaches to 70 preferably on the side 20a of torso 20 opposite the securing members 12 and 18.

### C. Re-Positioning Members

The elongated re-positioning members 22 and 24 operatively couple the arm securing member 12 to the torso securing member 18 for aligning or adjusting the position of the shoulder 28 and the scapula of the subject 16. The repositioning member 22 is preferably a flexible elongated strap or belt having ends 22a and 22b. The end 22a is preferably sewn or otherwise secured to arm member 42 in a non-removable fashion. A fastener 117 is attached on an underside 120 of the re-positioning member 22 to allow the re-positioning member 22 to attach to the exterior side 44a of arm number 44. The end 22b includes a re-adjustable fastener 118, preferably of the hook-type, positioned on the underside 120 of the elongated re-positioning member 22. It is preferable that the end 22b removably attaches to the extension member 68. Alternatively, the end 22b can be removably attached to the extension member 70 instead of the extension member 68. The elongated re-positioning member 22 functions as a re-alignment or re-positioning member by applying a re-positioning force to the shoulder 28 of the subject 16. This re-positioning force can include: (a) a rearward or z-axis line of force 96 which pulls the shoulder 28 from the front of the subject 16 to the back of the subject 16 which can also stabilize the scapula against the thoracic wall of the subject 16; (b) an upward or y-axis line of force 122 which pulls the arm 14 and shoulder 28 upward; and/or (c) an inward or x-axis line of force 60 which compresses the shoulder 28 into the body of the subject 16. Accordingly, the re-positioning member 22 has a rearward line of force 96, and upward line of force 122 and an inward line of force 60. The re-positioning member 22 can thereby dynamically: (a) re-position the shoulder 28 and the scapula from abnormal z-axis positions to normal or more normal z-axis positions; (b) redistribute part of the weight or load of the shoulder 28 and arm 14 to the torso 20 of the subject 16; and (c) laterally re-position the shoulder 28 to a normal or more normal position.

The re-positioning member 24 is preferably a strap or belt with relatively reduced elasticity having ends 24a and 24b. The end 24a is preferably sewn or otherwise non-removably affixed to arm member 44. The end 24b includes a suitable re-adjustable fastener 124, preferably of the hook-type, attached to the underside 126 of the repositioning member 24. The elongated member 24 preferably extends horizontally across the rear torso portion 20 and is of sufficient length to adjustably and removably attach to a portion of extension member 52. The elongated member 24 applies one or more re-positioning forces to rotate the shoulder 28 backward, pull the shoulder 28 backward or compress the shoulder 28 and the scapula into the body of the subject 16. These re-positioning forces include an x-axis line of force and a z-axis line of force. These lines of forces, coupled with the lines of forces 60, 122 and 96 of the elongated re-positioning member 22, facilitate the re-positioning of the shoulder and the scapula from an abnormal position to a normal or more normal position. The action of the re-positioning members 22 and 24 also preferably reduces pain to subjects afflicted with shoulder or scapula medical conditions. Furthermore, re-positioning members 22 and 24 preferably reduce or prevent overstretching of the serratus anterior muscle of such subjects.

### D. Engagement Member

The engagement member 26 of the position adjustment device 10 is preferably a rigid buckle loop sewn or otherwise attached to a side 126 of arm member 44 and to a side 128 of the rear torso engagement member 64. The engagement member 26 is preferably secured to a plurality of stitches (not shown) which connect the side 126 of the arm member 44 to the side 128 of the rear torso engagement member 64. The engagement member 26 receives the end 526 of the extension member 52 for the re-positioning and compression purposes of the arm member 44 described above. The extension member 52 adjustably passes through the engagement member 26 and folds over onto itself to reversibly and removably attach to itself by the re-adjustable fastener 56.

### E. Materials

The securing members 12 and 18 can be constructed of an integral single-piece garment including a relatively soft and flexible fabric or material. It is preferable that the material used to construct securing members 12 and 18 has elastic or resilient characteristics which provide additional re-positioning forces when the position adjustment device 10 is installed.

It is preferable that the material used to construct securing members 12 and 18 re-positioning member 22, and the comfort member 54 of the extension member 52 is two-ply or otherwise has a plurality of layers 86 and 88, as described above with respect to the rear torso engagement member 64 of the securing member 18. The material of the outer layer 86 is preferably made of an unbroken nylon loop-type or UBL material that is relatively lightweight and is relatively resistant to moisture. The UBL, which preferably covers the entire outer layer 86, facilitates the installation and adjustment of the position adjustment device 10 of the present invention.

The material of the inner layer 88 is also relatively lightweight and preferably resistant to moisture. The inner layer 88 preferably defines a plurality of relatively uniformly distributed small openings or orifices. These orifices help expose the skin to air for ventilation purposes. It is also preferable that the inner layer 88 has a hypoallergenic characteristic which enables the inner layer 88 to frictionally interact with the skin to hold the position of the position adjustment device 10 on the engaged body portion. One suitable material for the inner layer 88 is a perforated neoprene material defining orifices having a size in the range of one and one-half to two millimeters, preferably two millimeters. It should be appreciated that various open cell materials can be used as the material for the inner layer 88.

Any suitable flexible material or fabric can be used to construct the extension member 52 and the re-positioning member 24. It is preferable that extension member 52 and re-positioning member 24 are constructed of a relatively non-elastic, flexible material covered with a uniformly applied loop or UBL material compatible with a re-adjustable hook-type fastener.

As described above, the stiffening member 66 is preferably constructed of a plastic or polymer, however the stiffening member 66 can be constructed of any suitable material that has sufficient integrity to resist deformation.

The re-adjustable fasteners used in the position adjustment device 10 can include any suitable connectors which enable a plurality of parts to securely, yet removably, engage with one another. Such fasteners are preferably made of the hook-type material, although snaps, zippers and other suitable fastener can be used. The hook portion of Velcro®, a commercially available fastener, is one suitable hook-type fastener which can be used in the position adjustment device 10. The non-removable or permanent-type fasteners used in the position adjustment device 10 can include any suitable fastener or fastening technique which securely and non-removably attaches one part to another part, including, without limitation, stitches, threads, adhesives, molding, pins and zippers.

It should also be appreciated that part or all of the position adjustment device 10 can also be constructed of a network of rubber cords, plastic hooks and/or cotton fabric.

### F. Installation

When installed, the position adjustment device 10 generally conforms to the configuration illustrated in FIGS. 1 to 2 and is adjustable by a user to re-position the shoulder and scapula from an abnormal position to a normal or more normal position. The user first positions the securing members 12 and 18 on his or her respective body portions 14 and 20b. Next, the user: (a) adjustably attaches the re-adjustable fastener 50 of arm member 44 to arm member 42; (b) adjustably attaches the re-adjustable fastener 51 of arm member 46 to arm member 48; and (c) adjustably attaches the re-adjustable fastener 78 of the front torso engagement member flap 76 to the rear torso engagement member flap 80. The user then secures the securing members 12 and 18 to the torso by wrapping securing members 68 and 70 horizontally around the front and back of the torso adjustably attaching securing member 68 to securing member 70 on the side of the torso 20a.

The user then preferably positions and adjusts the tension of the extension member 52. The user preferably positions extension member 52 to compress the shoulder 28 into the shoulder joint by wrapping the extension member 52 across the front of the torso 20 and under the arm 58. The user further directs the strap across the back toward the engagement member 26 where the end 52b of the re-positioning member 52 passes through the loop of the engagement member 26 and adjustably folds over itself to allow the fastener 56 of the re-positioning member 52 to attach to the re-positioning member 52. To further re-position the shoulder, the user adjustably attaches the elongated member 24 to a location along the length of re-positioning member 52, the user can rotate or pull the shoulder 28 in a rearward direction. The user then preferably attaches the fastener 117 of re-positioning member 22 to the exterior surface 44a of arm member 44 and attaches the fastener 118 of the re-positioning member 22 to the securing members 68 or 70 in order to align the shoulder 28 near or adjacent to the ear 130 by pulling the arm 14 backwards in relation to the subject 16.

In the example illustrated in FIGS. 1 to 8, the position adjustment device 10 is shown as a left-sided version which preferably treats the subject's 16 left shoulder 28 and left scapula. It should be appreciated that, in other examples not illustrated, the adjustment device 10 can be adapted to treat the subject's right shoulder and right scapula. In addition, the position adjustment device 10 can be constructed in various sizes and colors suitable for children and adults.

Although the position adjustment device 10 of the present invention is preferably used for re-positioning shoulder 28 to a backward position from an abnormal forward thrust position, the position adjustment device 10 can be adapted to re-position the shoulder 28 or other portions of the body from any abnormal position to a normal or more normal position, including, without limitation, shoulders and limbs which have an abnormal backward thrust position.

The position adjustment device 10 of the present invention provides adduction and other re-positioning of a wearer's shoulder joint and scapula by pushing or compressing the shoulder and scapula into the body. Specifically, the position adjustment device 10 moves the wearer's shoulder 28 from an abnormal front position to normal or more normal rearward position in order to increase proper alignment of the wearer's shoulder. The position adjustment device 10 also flattens out a protruding scapula by compressing the scapula with a relatively flat stiffening member. Also, the position adjustment device 10 transfers part of the wearer's arm and shoulder load to the wearer's torso or lower back. When the wearer moves his/her arm and shoulder, such as when throwing a ball, the position adjustment device 10 helps keep the wearer's shoulder joint in socket while enabling the wearer to move his/her arm 14 through a relatively broad range of motion. It is preferable that the position adjustment device 10 has the effects of stabilizing the scapula against the thoracic wall, reducing or preventing pain and reducing or preventing overstretching of the serratus anterior muscle, improving active shoulder control and strength through scapula support, and playing a therapeutic role in the rehabilitation of the shoulder joint. This type of device provides wearers with dynamic bodily support during daily activity to improve and enhance their quality of life.

### II. Second Embodiment

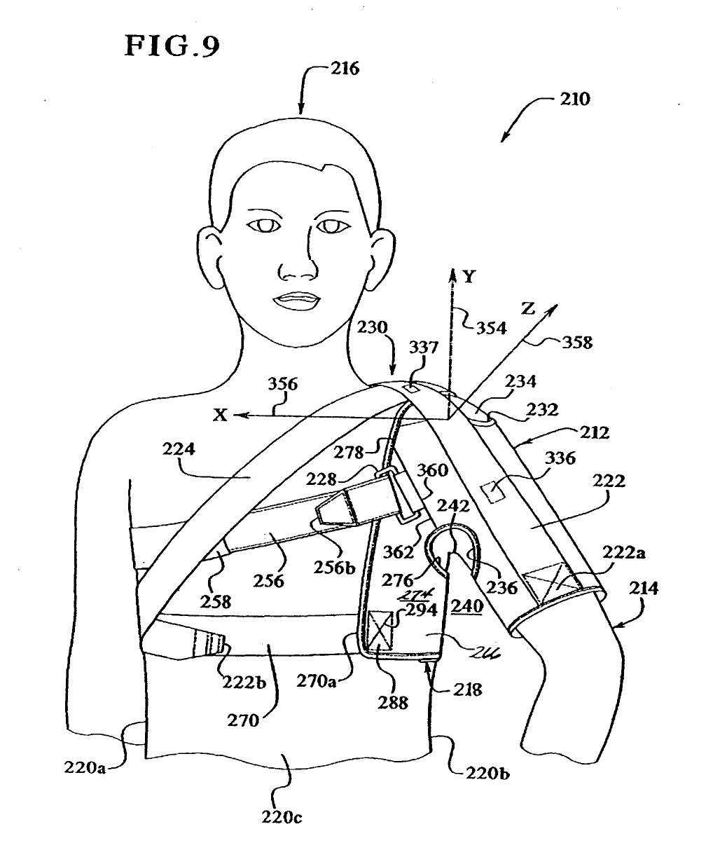

As illustrated in FIGS. 9 to 14, in one embodiment the position adjustment device 210 includes: (a) an arm securing member 212 which is secured to an abnormally positioned upper arm 214 or other body portion of a subject 216; (b) a torso securing member 218 which is engaged with the subject's upper torso 220 or other body portion which preferably serves as an anchor; (c) one or more elongated re-positioning members 222, 224 and 226 coupled to the arm securing member 212 and the torso securing member 218 as described below; and (d) one or more engagement members 228 connected to both the arm securing member 212 and the torso securing member 218.

### A. Arm Securing Member

The arm securing member 212 is preferably an arm holder or sleeve member which is configured to have a tubular shape fitting around the upper arm 214 and shoulder 230 area of the subject 216. It is also preferable that the arm securing member 212 includes a shoulder wall 232 which defines an adjustment slot, flex slot or shoulder opening 234. The shoulder wall 232 receives the protruding, substantially round shoulder 230. Accordingly, the shoulder wall 232 facilitates the secure attachment of the arm securing member 212 to the shoulder 230 when the subject 216 moves his/her shoulder to various positions.

In addition, the arm securing member 212 preferably includes a plurality of under arm walls 236 and 238 which define an armpit cavity or under arm cavity 240. The under arm cavity 240 allows the under arm surface 242 of the subject 216 to receive ventilation. The under arm cavity 240 also enhances the comfort of the subject 216 and facilitates the free movement of the subject's arm 214 when the subject 216 is wearing the position adjustment device 110.

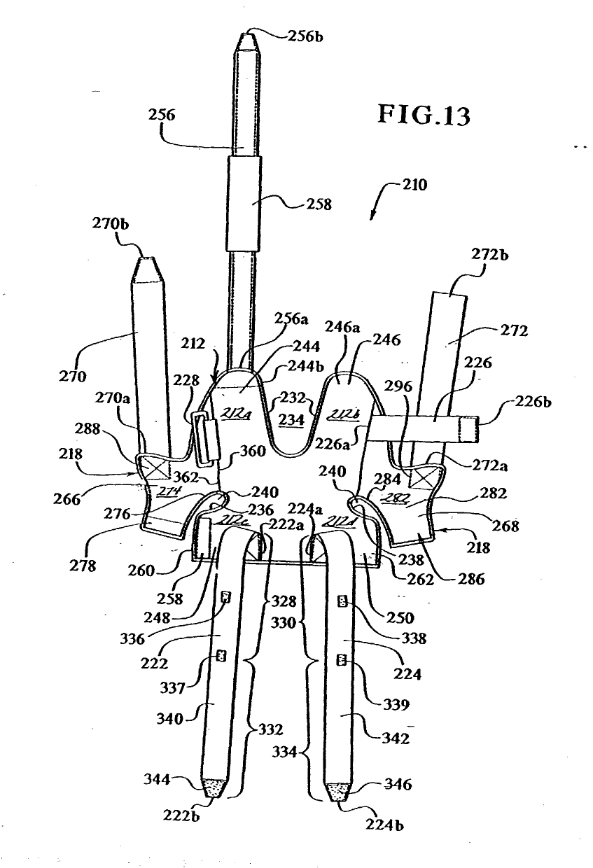

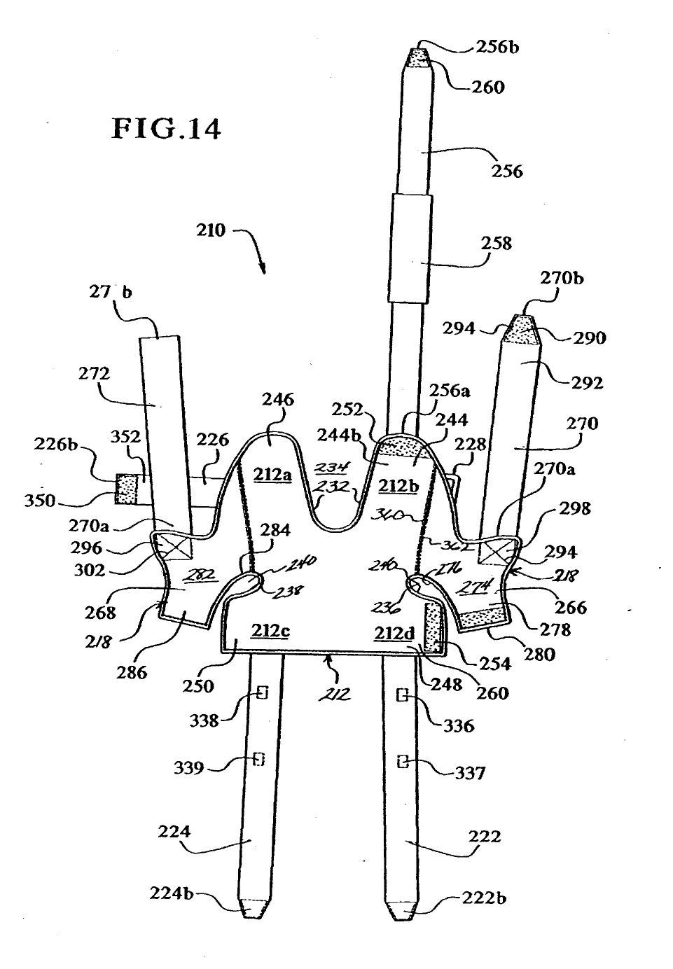

As best shown in FIGS. 13 and 14, the arm securing member 212 preferably has four arm members 244, 246, 248, and 250. Arm member 244 is located in the upper (or superior) and front (or anterior) portion 212a of the arm securing member 212. Arm member 246 is located in the upper (or superior) and rear (or posterior) portion 212b of the arm securing member 212. Arm member 246 is located in the lower (or inferior) and front (or anterior) portion 212c of the arm securing member 212. Arm member 250 is located in the lower (or inferior) and rear (or posterior) portion 212d of arm securing member 212. It is preferable that the arm member 244 includes a re-adjustable fastener 252, such as a hook-type fastener, attached on the under side 244b of the arm member 244. When installed on the subject 216, the arm member 244 preferably intersects with and removably attaches to the outer or exterior side 246a of the arm member 246. In this configuration, arm members 244 and 246 encompass the upper area of the shoulder 230 of the subject 216.

It is also preferable that arm member 248 includes a re-adjustable fastener 254 such as a hook-type fastener, attached on the under side 260 of the arm member 248. When installed on the subject 216, arm member 248 preferably intersects with and removably attaches to the outer or exterior side 250a of the arm member 250. In this configuration, arm members 248 and 250 encompass the upper arm 214 of the subject 216.

Furthermore, the arm member 244 preferably includes an elongated member 256 having: (a) ends 256a and 256b; (b) a cushioning or comfort member 258 positioned between ends 256a and 256b; and (c) a re-adjustable fastener 260, preferably of a hook-type. The end 256a is non-removably attached to the upper portion of arm member 244. When installed, the elongated member 256 extends across the upper torso 220 of the subject 216, and the comfort member 258 is positioned under an arm 262 opposite the arm 214 of the subject 216. The end 256b removably engages with the engagement member 228 as described below. By adjusting the engagement of the elongated member 256 to the engagement member 228, different degrees of pressure or lines of force 264 can be applied in order to compress and support the shoulder 230 in a normal or more normal position. Accordingly, the extension member 256 functions as a compression and support member, causing the shoulder 230 to be compressed into the body of the subject 216, preferably into the shoulder girdle (not shown) so as to align the humeral head to the glenoid fossa.

### B. Torso Securing Member

The torso securing member 218, which is coupled to the arm securing member 212, preferably includes a structure that is configured to engage at least a portion of the upper torso 220 of subject 216. It is preferable that the torso securing member 218 includes: (a) a front (or anterior) torso engagement member 266; (b) a rear or back side (or posterior) torso engagement member 268; and (c) one or more straps, or securing members 270 and 272 connected to torso engagement members 266 and 268, respectively.

It is preferable that the torso engagement members 266 and 268 are sewn or otherwise secured to the arm members 244 and 246, respectively, in a non-removable fashion. The front torso engagement member 266, connected to arm member 244, includes: (a) a relatively flat chest or abdomen engagement member 274; (b) an under arm wall 276 further defining the under arm cavity 240; and (c) a rear extension flap 278 having a re-adjustable fastener 280 which removably fastens to the rear torso engagement member 268. The flap 278 allows the front torso engagement member 266 to removably attach to the rear torso engagement member 268 under the arm 214 of the subject 216. The under arm wall 276, which further defines under arm cavity 240, reduces restraints on movement of the arm 114 and also enhances the comfort and ventilation of the subject 116.

The rear torso engagement member 268, connected to arm member 246, includes: (a) a relatively flat back side (or posterior) torso engagement member 282 which is secured to the back side 220 of the subject 216; (b) an under arm wall 284 further defining the under arm cavity 240; and (c) an extension or flap 286 which removably connects to the flap 278 of the front torso engagement member 266.

It is preferable that a reinforcement member 298 be attached to the edges of the securing members 212 and 218 to protect the layers of material of the position adjustment device 210 from separating, tearing, fraying or otherwise being damaged and to aid in reinforcing and maintaining the shape of the brace. The reinforcement member 298 is preferably a binding or trim made of a flexible fabric or material having a relatively narrow uniform width sufficient to engage a portion of both outer and inner layers of the securing members 212 and 218. The reinforcement member 298 engages the surface of the outer layer of the securing members 212 and 218 and is wrapped over the edges of the outer and inner layers to engage the surface of the inner layer edge. The reinforcement member 298 is preferably non-removably secured to the surfaces of the outer and inner layers and by a plurality of stitches. The reinforcement member 298 preferably has a length sufficient to form a continuous edge along the contours of the securing members 212 and 218. It should be appreciated that other devices and techniques can be used to reinforce the attachment of the inner layer to the outer layer of the securing members 212 and 218.

The securing members 270 and 272 of the torso securing member 218 preferably include adjustable straps or elongated securing members which secure the torso engagement members 266 and 268 to the torso 220 of the subject 216. Securing member 270 has two ends 270a and 270b. The end 270a is attached in a non-removable fashion to a lower portion 288 of the front torso engagement member 266 and is oriented substantially horizontally across the torso 220 of the subject 216. The free end 270b of securing member 270 includes a re-adjustable fastener 290. The fastener 290 is preferably secured to the underside 292 of end 270b using a plurality of stitches 294.

Similarly, securing member 272 has two ends 272a and 272b. The end 272a is non-removably attached to a lower portion 296 of the torso engagement member 268 and is oriented substantially horizontally across the torso 220 of the subject 216. The free end 272b of securing member 272 is positioned to receive the end 270b of the securing member 270. Securing members 270 and 272 connect the front and back torso engagement members 266 and 268 to the side 220a of the torso 220 and are preferably configured to wind or wrap around opposite sides of the torso 220 toward one another. Specifically, securing member 270 preferably wraps around the front side 220c of torso 220, and securing member 272 preferably wraps around the back side 220d of torso 220. The securing member 270 overlaps and removably attaches to 272 preferably on the side 220a of torso 220 opposite the torso securing member 218.

### C. Repositioning Members

The re-positioning members 222 and 224 work together with re-positioning member 226 to operatively couple the arm securing member 212 to the torso securing member 218 for aligning or adjusting the position of the shoulder 230 and also for reducing load on shoulder tissue. The re-positioning members 222 and 224 are preferably flexible elongated straps or belts. The re-positioning member 222 has ends 222a and 222b, and the re-positioning member 224 has ends 224a and 224b. The ends 222a and 224a are preferably sewn or otherwise secured to the lower ends of the arm members 248 and 250, respectively, in a non-removable fashion. End 222a is, therefore, preferably in front of the arm 214, and end 224a is in the rear of the arm 214.

It is preferable that each re-positioning member 222 and 224 is divided into two segments along its lengths: arm or shoulder segments 328 and 330, respectively and cross-body or torso segments 332 and 334, respectively. Each re-positioning member 222 and 224 is preferably directed upward toward the shoulder. Readjustable fasteners 336 and 338, sewn or otherwise secured to the underside of the re-positioning members 222 and 224, respectively, attach the re-positioning members 222 and 224 to the arm securing member 212 approximately halfway between the origin of the re-positioning members 222 and 224 and the shoulder. The repositioning member 222 further extends over the shoulder 230 where the re-positioning members 222 and 224 cross and are secured to arm member 244 by a re-adjustable fastener 337, preferably of the hook-type, positioned on the underside 340 of the re-positioning member 222. An additional re-adjustable fastener 339 positioned on the underside 342 of the re-positioning member 224 secures the re-positioning member 224 to re-positioning member 222 where they intersect at the top of the shoulder 230. It should be appreciated that each of the re-positioning members 222 and 224 can be removably attached to the shoulder 230 and to one another in any sequence with the re-positioning member 222 on top of the re-positioning member 224 or vice-versa.

From the attachment point on the top of the shoulder 230, the re-positioning member 222 preferably extends across the back 220d of the subject 216 and downward to reversibly attach to securing members 270 or 272 of the torso securing member 218. In similar fashion, the re-positioning member 224 intersects the re-positioning member 222 and extends across the front 220c of the subject 216 where a readjustable fastener 339 attaches to the elongated member 256. The repositioning member 224 further extends downward to reversibly attach to the securing members 270 or 272 of the torso securing member 218.

The ends 222b and 224b include re-adjustable fasteners 344 and 346, respectively, preferably of the hook-type, positioned on the undersides 340 and 342, respectively, of re-positioning members 222 and 224. It is preferable that the ends 222b and 224b removably attach to the securing members 270 or 272.

The re-positioning members 222 and 224 function as compression members and are load re-distribution members as described below. The shoulder segments 328 and 330 of re-positioning members 222 and 224 act in concert to re-align or re-position the arm in relation to the shoulder joint by applying a balanced re-positioning force to the arm 214 of the subject 216. This re-positioning force can include: (a) an upward line of force 354 along a y-axis which pulls the arm 214 upward into the shoulder joint 230, preferably aligning the humeral head to the glenoid fossa; and/or (b) an inward line of force 356 along an x-axis which compresses the shoulder 230 into the shoulder socket so as to reduce subluxation. A forward z-axis line of force (not shown) produced by the re-positioning member 222 is substantially counter-balanced by a rearward z-axis line of force (not shown) produced by the re-positioning member 224 to control movement in either direction along the z-axis. Accordingly, the re-positioning members 222 and 224 have an upward line of force 354 and an inward line of force 356. The re-positioning members 222 and 224 can thereby dynamically: (a) re-position the arm 214 to a normal or more normal position in the shoulder joint; and (b) redistribute part of the weight or load of the shoulder 230 and arm 214 to the torso 220 of the subject 216.

The re-positioning member 226 is preferably a strap or belt with relatively reduced elasticity having ends 226a and 226b. The end 226a is preferably sewn or otherwise attached to the side of arm member 246. The end 226b includes a suitable removable fastener 350, preferably of the hook-type, positioned on the underside 352 of the re-positioning member 226. The re-positioning member 226 preferably extends horizontally across the rear torso portion 220 and is of sufficient length to adjustably and removably attach to a portion of elongated member 256. The re-positioning member 226 applies a re-positioning force 358 to rotate the shoulder 230 backward or pull the shoulder 230 backward and to compress the shoulder 230 into the shoulder socket of the subject 216. The re-positioning force 358 is coupled with the lines of forces 354 and 356 of re-positioning members 222 and 224. All of the lines of forces 354, 356, and 358 facilitate the re-positioning of the shoulder 230 from an abnormal position to a normal or more normal position. It is preferable that the action of re-positioning members 222, 224 and 226 dynamically supports the shoulder joint, aligns the humeral head to the glenoid fossa, reduces pain, decreases load on tissues and reduce or prevents subluxation.

### D. Engagement Member

The engagement member 228 of the position adjustment device 210 is preferably a rigid buckle loop sewn or otherwise attached to a side 360 of arm member 244 and to a side 362 of the torso engagement member 276. The engagement member 228 is preferably secured to a plurality of stitches (not shown) which connect the side 360 of the arm member 244 to the side 362 of the torso engagement member 266. The engagement member 228 receives the end 256b of the elongated member 256 for the compression purposes of the arm member 244 described above. The elongated member 256 adjustably passes through the engagement member 228 and folds over onto itself to reversibly and removably attach to itself by the re-adjustable fastener 260.

### E. Materials

The securing members 212 and 218 can be constructed of an integral single-piece garment including a relatively soft and flexible fabric or material. It is preferable that the material used to construct securing members 212 and 218 has elastic or resilient characteristics which provide additional re-positioning forces when the position adjustment device 210 is installed.

It is preferable that the material used to construct securing members 212 and 218 re-positioning members 222 and 224, and the comfort member 258 of the elongated member 256 is two-ply or otherwise has a plurality of layers. The material of the outer layer is preferably made of an unbroken nylon loop-type or UBL material that is relatively lightweight and is relatively resistant to moisture. The UBL, which preferably covers the entire outer layer, facilitates the installation and adjustment of the position adjustment device 210 of the present invention.

The material of the inner layer is also relatively lightweight and preferably resistant to moisture. The inner layer preferably defines a plurality of relatively uniformly distributed small openings or orifices. These orifices help expose the skin to air for ventilation purposes. It is also preferable that the inner layer has a hypoallergenic characteristic which enables the inner layer 88 to frictionally interact with the skin to hold the position of the position adjustment device 210 on the engaged body portion. One suitable material for the inner layer is a perforated neoprene material defining orifices having a size in the range of one and one-half to two millimeters, preferably two millimeters. It should be appreciated that various open cell materials can be used as the material for the inner layer.

Any suitable flexible material or fabric can be used to construct the elongated member 256 and the re-positioning member 226. It is preferable that elongated member 256 and re-positioning member 226 are constructed of a relatively non-elastic, flexible material covered with a uniformly applied loop or UBL material compatible with a re-adjustable hook-type fastener.

The re-adjustable fasteners used in the position adjustment device 210 can include any suitable connectors which enable a plurality of parts to securely, yet removably, engage with one another. Such fasteners are preferably made of the hook-type material, although snaps, zippers and other suitable fastener can be used. The hook portion of Velcro®), a commercially available fastener, is one suitable hook-type fastener which can be used in the position adjustment device 210. The non-removable or permanent-type fasteners used in the position adjustment device 210 can include any suitable fastener or fastening technique which securely and non-removably attaches one part to another part, including, without limitation, stitches, threads, adhesives, molding, pins and zippers.

It should also be appreciated that part or all of the position adjustment device 210 can also be constructed of a network of rubber cords, plastic hooks and/or cotton fabric.

### F. Installation

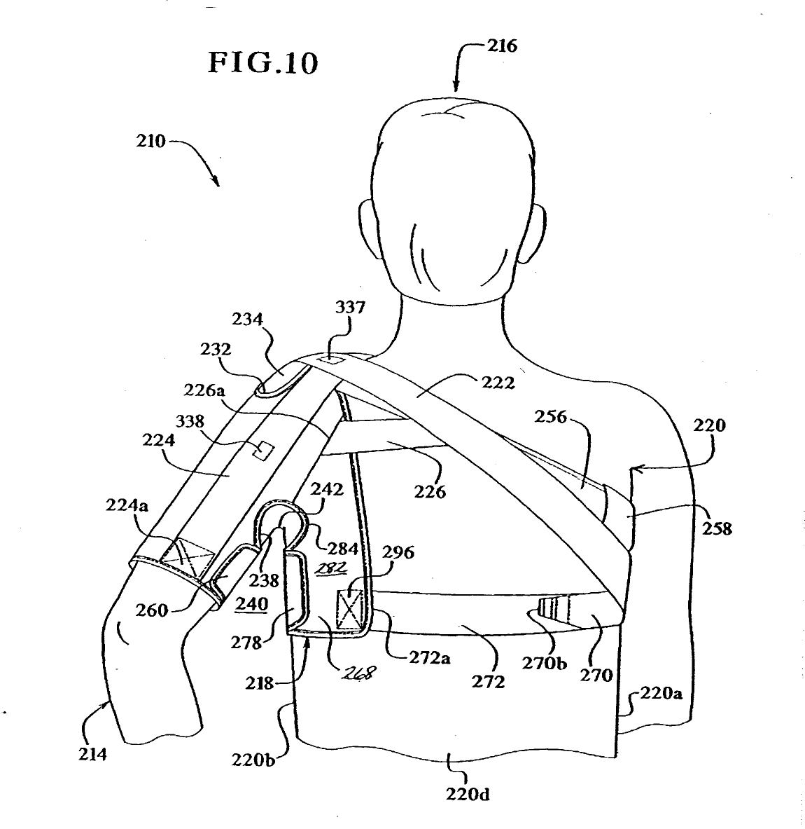

The position adjustment device 210 generally conforms to the configuration illustrated in FIGS. 9 and 10 and is adjustable by a user to re-position the shoulder 230 from an abnormal position to a normal or more normal position. The user first positions the securing members 212 and 218 on their respective body portions 214 and 220. Next, the user: (a) adjustably attaches the fastener 254 of arm member 248 to the exterior side 250a of the arm member 250; (b) adjustably attaches the fastener 252 of arm member 244 to the exterior side 246a of arm member 246; (c) adjustably attaches elongated member 256 to the engagement member 228; (d) adjustably attaches the fastener 280 of the front torso extension flap 278 to the rear torso extension flap 286 of the rear torso engagement member 268; and (e) adjustably attaches the securing members 270 and 272 to one another. The user then preferably positions and adjusts the tension of the re-positioning member 226 to elongated member 256. Next, the user positions and adjusts the tension of the re-positioning members 222 and 224. The user preferably positions segments 328 and 330 of re-positioning members 222 and 224 to compress the shoulder 230 into the shoulder joint reducing or preventing subluxation or dislocation of the shoulder 230. Also, the user preferably crosses the re-positioning members 222 and 224 at the shoulder attaching one to the other. Next, the user directs segments 332 and 334 of the re-positioning members 222 and 224, respectively, from the shoulder 230 to the side 220a of the torso 220 in order to stabilize the shoulder 230 in relation to the body of the subject 216.

In the example illustrated in FIGS. 9 to 14, the position adjustment device 210 is shown as a left-sided version which preferably treats the subject's left shoulder 230. It should be appreciated that, in other examples not illustrated, the adjustment device 210 can be adapted to treat the subject's right shoulder. In addition, the position adjustment device 210 can be constructed in various sizes and colors suitable for children and adults.

Although the position adjustment device 210 of the present invention is preferably used for re-positioning a subluxated shoulder 230 to a position within the glenohumoral joint (not shown) and to a normal or more normal rearward position, the position adjustment device 210 can be adapted to re-position the shoulder 230 or other portions of the body from any abnormal position to a normal or more normal position, including, without limitation, shoulders and limbs which have an abnormal dislocated or backward thrust position.

The position adjustment device 210 of the present invention moves the wearer's arm 214 from an abnormally aligned position to a normal or more normal aligned position. The position adjustment device 210 also moves and supports the wearer's shoulder joint by pushing or compressing the wearer's shoulder into the shoulder socket. Also, the position adjustment device 210 transfers part of the wearer's arm and shoulder load to the wearer's torso or lower back. When the wearer moves his/her arm and shoulder, such as when throwing a ball, the position adjustment device 210 helps keep the wearer's shoulder joint in socket while enabling the wearer to move his/her arm through a relatively broad range of motion. It is preferable that the position adjustment device 210 has the effects of supporting the shoulder joint, aligning or aiding in the alignment of the humeral head to the glenoid fossa, reducing or preventing pain, decreasing load on tissues, reducing or eliminating subluxation of the shoulder joint and playing a therapeutic role in the rehabilitation of the shoulder joint. This type of device provides wearers with dynamic bodily support during daily activity to improve and enhance their quality of life.

### III. Third Embodiment

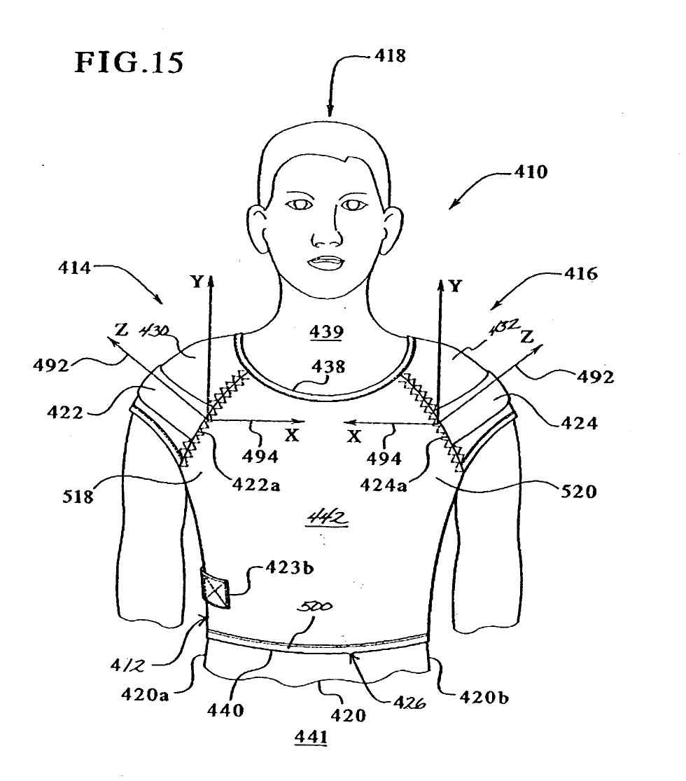

As illustrated in FIGS. 15 to 19, in one embodiment the position adjustment device 410 includes: (a) a torso securing member 412 which is engaged with the torso 420 and right and left shoulders 414 and 416 or other body portions of the subject 418; and (b) one or more re-positioning members 422, 423 and 424 which connect a front portion 426 of the torso securing member 412 to a rear portion 428 of the torso securing member 412.

### A. Torso Securing Member

The torso securing member 412 preferably includes: (a) the front (or anterior) torso engagement member 426; (b) the rear (or posterior) torso engagement member 428; (c) shoulder engagement members 430 and 432 that join in a non-removable fashion the front torso engagement member 426 with the rear torso engagement member 428; (d) under arm walls 434 and 436 defining an arm opening 435 and 437 respectively; (e) a neck wall 438 defining a neck opening 439; and (f) a lower torso wall 440 defining a torso opening 441.

The front torso engagement member 426 includes: (a) a relatively flat chest and/or abdomen engagement member 442 which is preferably configured to cover the front torso area 420 of the subject 418; and (b) extension members or flaps 444 and 446 for connecting the front torso engagement member 426 to the rear torso engagement member 428 described below. The flaps 444 and 446 of the front torso engagement member 426 wrap rearwardly around torso sides 420a and 420b, respectively, to adjustably and removably attach to the rear torso engagement member 428. Re-adjustable fasteners 448 and 450, preferably of the hook-type, are attached to flaps 444 and 446, respectively, and engage the rear torso engagement member 428.

The rear torso engagement member 428 of the torso securing member 412 includes a right rear torso engagement member 468 and a left rear torso engagement member 470. The right rear torso engagement member 468 is joined to the left rear torso engagement member 470 by a re-adjustable fastener 476 attached to a center flap 472 of the right rear torso engagement member 468. Fastener 476 removably attaches to a center flap 474 of the left rear torso engagement members 468. Alternatively, it should be appreciated that the readjustable fastener 476 can be attached to the center flap 474 of the left torso engagement member 470. The right and left rear torso engagement members 468 and 470 preferably include extension members or side flaps 478 and 480, respectively. Flaps 478 and 480 preferably extend around the sides 420a and 420b of the torso 420 of the subject 418. Flap 478 removably attaches to the re-adjustable fastener 448 of the flap 444 of the front torso engagement member 426, and flap 480 removably attaches to the re-adjustable fastener 450 of the flap 446 of the front torso engagement member 426.

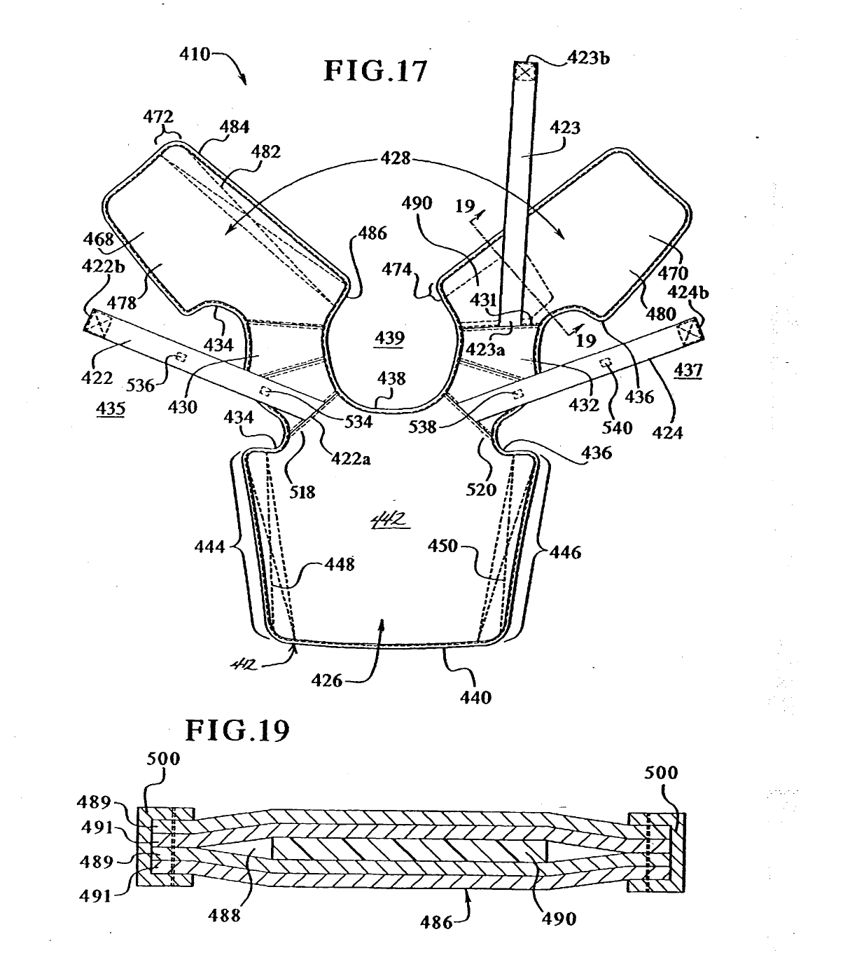

The position adjustment device 410 preferably has an outer layer 489 and an inner layer 491. As best shown in FIGS. 17 and 19, the left rear torso engagement member 470 preferably includes an additional panel 486 non-removably secured to a portion of the inner layer 491 of the left rear torso engagement member 470.

The left rear torso engagement member 470 includes the panel 486 which is oriented such that the outer layer 489 of the panel is against the inner layer 491 of the left rear torso engagement member 470 defining a space 488 in which the stiffening member 490 is contained. The orientation of panel 486 allows the panel 486 to position the inner layer 491 against the skin of the subject 416. The stiffening member 490 preferably has a relatively flat shape and is positioned and sized to fill the space 488. The stiffening member 490 is preferably relatively flat in shape and positioned and sized to substantially fill a space between the outer wall 489 and inner wall 491. In this position, the stiffening member 490 provides a pivot point or support point against which re-positioning members 422 and 423 apply a line of force 492. The stiffening member 490 contained within the left rear torso engagement member 470 resists a rearward pressure or line of force 492 acting on an abnormally positioned scapula. As a result, such forces can align or aid in the alignment of the left scapula relative to the rib cage. It should be appreciated that the position adjustment device 410 can be adapted to include a right scapula stiffening member in addition to or instead of the left scapula stiffening member 490.

The shoulder engaging members 430 and 432 wrap over the surface of the shoulders 414 and 416, respectively. The shoulder engaging members 430 and 432 are each non-removably attached to the front torso engagement member 426 and the rear torso engagement member 428.

It is preferable that a reinforcement member 500 be attached to the edges of the torso securing member 412 to protect the layers of material of the position adjustment device 410 from separating, tearing, fraying or otherwise being damaged and to aid in reinforcing and maintaining the shape of the brace. The reinforcement member 500 is preferably a binding or trim made of a flexible fabric or material having a relatively narrow uniform width sufficient to engage a portion of both outer and inner layers 489 and 491 of the torso securing member 412. The reinforcement member 500 engages the surface of the outer layer 489 edge of the torso securing member 412 and is wrapped over the edges of the outer and inner layers 489 and 491 to engage the surface of the inner layer 491 edge. The reinforcement member 500 is preferably non-removably secured to the surfaces of the outer and inner layers 489 and 491 by a plurality of stitches. The reinforcement member 500 preferably has a length sufficient to form a continuous edge along the contours of the torso securing member 412. It should be appreciated that other devices and techniques may be used to reinforce the attachment of the inner layer 491 to the outer layer 489 of the torso securing member 412.

### B. Re-Positioning Members

The re-positioning members 422 and 424 are preferably flexible members or straps. Re-positioning member 422 has ends 422a and 422b and repositioning member 424 has ends 424a and 424b. Ends 422a and 424a are preferably sewn or otherwise secured to upper positions 518 and 520 of the front torso engaging member 426 in a non-removable fashion. Ends 422a and 424a are positioned on the front torso engaging member 426 to allow re-positioning members 422 and 424 to engage a sufficient area of the shoulders 414 and 416 to apply a relatively wide range of re-positioning force to the shoulders 414 and 416. The re-positioning members 422 and 424 wrap around and engage the shoulders 414 and 416 and are directed at any suitable angle 522 from a horizontal axis 523. The re-positioning members 422 and 424 are preferably of a sufficient length to cross the vertical midline 524 of the back of subject 418.

The re-positioning member 422 includes hook-type fasteners 534 and 536 that are spaced along the length of the re-positioning member 422 to adjustably attach to the shoulder engagement member 430 and the right rear torso engagement member 468, respectively. Similarly, the re-positioning member 424 includes hook-type fasteners 538 and 540 that are spaced along the length of the re-positioning member 424 to adjustably attach to the shoulder engagement member 432 and the repositioning member 423 or the left rear torso engagement member 470, respectively.

Ends 422b and 424b preferably include hook-type fasteners 526 and 528 on the undersides 530 and 532 of the re-positioning members 422 and 424, respectively. It is preferable that the re-adjustable fastener 526 attached to the end 422b of the re-positioning member 422 adjustably and removably attaches to the left rear torso engagement member 470. Similarly, it is preferable that the re-adjustable fastener 528 attached to the end 424b of the re-positioning member 424 adjustably and removably attaches to the right rear torso engagement member 468. It can be appreciated that elongated member 422 can be positioned over or underneath elongated member 424 at the midline 524 of the back of subject 416.

The re-positioning members 422 and 424 can function together as re-alignment or re-positioning members by applying a re-positioning force to both shoulders 414 and 416 of the subject 418. This re-positioning force includes: (a) a rearward or z-axis line of force 492 which pulls the shoulders 414 and 416 from the front of the subject 418 to the back of the subject 418; and (b) an inward or x-axis line of force 540 which compresses the shoulders 414 and 416 into the body of the subject 418 in the direction of the midline 524. Accordingly, the re-positioning members 422 and 424 each have a rearward line of force 492 and an inward line of force 494. The re-positioning members 422 and 424 thereby dynamically re-position,the shoulders 414 and 416 from an abnormal front position to a back position, preferably while re-positioning the left scapula as described above.

The re-positioning member 423, preferably a flexible member or strap has ends 423a and 423b. The end 423a is preferably sewn or otherwise non-removably secured to a seam 542 joining the shoulder engaging member 432 to the left rear torso engagement member 470. The re-positioning member 423 is directed at a suitable angle downward from the rear of the shoulder 416 across a substantial portion of the stiffening member 490 contained within the left rear torso engagement member 470. The re-positioning member 423 is preferably of a sufficient length to cross the vertical midline 524 of the back of subject 418 directed toward the torso side 420a. The end 423b extends around the torso side 420a and removably attaches to the front torso engagement member 426. The end 423b includes a hook-type fastener 527 on an underside 529 of the re-positioning members 423. It should be appreciated that elongated member 423 can be positioned over or underneath elongated member 422 and 424 as it is directed across the back of subject 418.

The re-positioning member 423 can function as a re-alignment or re-positioning member by applying additional re-positioning force to a shoulder 416 and/or scapula which is particularly out of place or dysfunctional. This re-positioning force can include: (a) a rearward or z-axis line of force 492 which rotates the shoulder 416 from a forward position to a more rearward position; (b) an inward or x-axis line of force 494 which further compresses the shoulder 416 into the body of the subject 418 in the direction of the midline 524; and (c) a downward y-axis line of force (not shown) which pulls the shoulder downward. Accordingly, the re-positioning member 423 has a rearward line of force 492, an inward line of force 494 and a downward line of force. The re-positioning member 423 thereby aids in dynamically re-positioning the shoulder 416 and associated scapula from an abnormal position to a normal or more normal position.

In addition, the re-positioning member 423 together with the re-positioning member 422 preferably reinforces or supports the pivot point or support provided by the stiffening member 490. Accordingly, the re-positioning member 423 helps to stabilize the stiffening member 490. As a result, the re-positioning member 423 can further align or aid in the alignment of the left scapula relative to the rib cage. It should be appreciated that the position adjustment device 410 can be adapted to include a re-positioning member that supports a right scapula stiffening member.

### C. Materials

It is preferable that the torso securing member 412 is constructed of an integral, single piece garment including a relatively soft and flexible fabric or material. It is preferable that the material used to construct the torso securing member 412 has elastic or resilient characteristics which provide additional repositioning forces when the position adjustment device 410 is installed.

The material of the torso securing member 412 preferably has two-plies or layers 489 and 491 described above with respect to the rear left torso engagement member 470. The outer layer 489 is preferably made of an unbroken nylon loop-type or UBL material that is relatively lightweight and is relatively resistant to moisture. The UBL, which preferably covers the entire outer layer 489, facilitates the installation and adjustment of the position adjustment device 410 of the present invention.

The inner layer 491 preferably defines a plurality of relatively uniformly distributed small openings or orifices. These orifices help expose the skin to air for ventilation purposes. It is also preferable that the inner layer 491 has a hypoallergenic characteristic which enables the inner layer 88 to frictionally interact with the skin to hold the position of the position adjustment device 410 on the engaged body portion. One suitable material for the inner layer 491 is a perforated neoprene material defining orifices having a size in the range of one and one-half to two millimeters, preferably two millimeters. It should be appreciated that various open-cell materials can be used as the material for the inner layer 491.

In the example illustrated in FIGS. 15 to 19, the position adjustment device 410 is shown as having a left-sided stiffening member which preferably treats the left scapula of subject 418. It should be appreciated that, in other examples not illustrated, the adjustment device 410 can be adapted to include one or more suitable stiffening members in order to treat either or both scapulas of the subject. The stiffening member 490, housed within the left rear torso engagement member 470 is preferably made of a plastic material or other material resistant to moisture that has sufficient integrity to resist deformation.

The re-adjustable fasteners used in the position adjustment device 410 can include any suitable connectors which enable a plurality of parts to securely, yet removably, engage with one another. Such fasteners are preferably made of the hook-type material, although snaps, zippers and other suitable fasteners can be used. The hook portion of Velcro®, a commercially available fastener, is one suitable hook-type fastener which can be used in the position adjustment device 410.

The non-removable or permanent-type fasteners used in the position adjustment device 410 can include any suitable fastener or fastening technique which securely and non-removably attaches one part to another part, including, without limitation, stitches, threads, adhesives, molding, pins and zippers.

It should also be appreciated that part or all of the position adjustment device 410 can also be constructed of a network of rubber cords, plastic hooks and/or cotton fabric.

### D. Installation

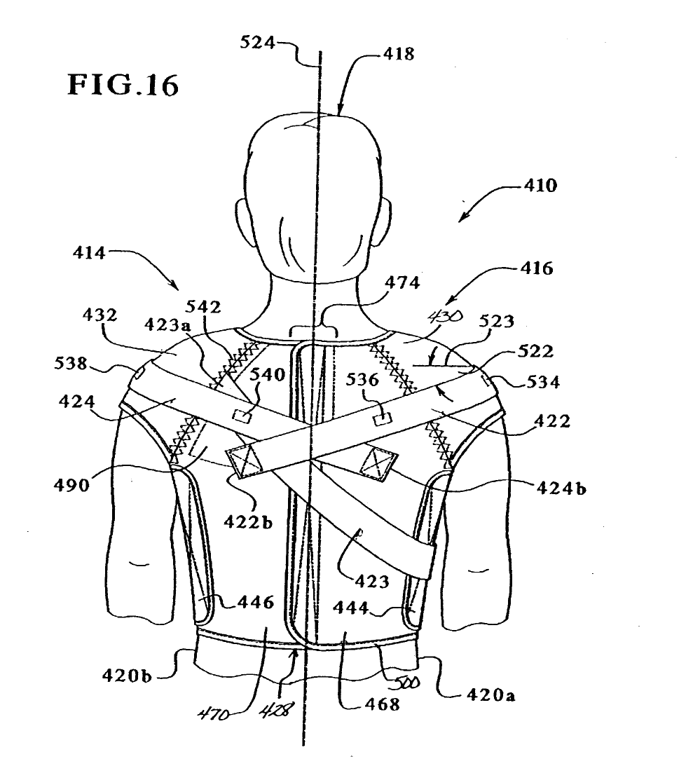

The position adjustment device 410 generally conforms to the configuration illustrated in FIGS. 15 and 16 and is adjustable by a user to re-position both shoulders and, in the illustrated example, the left scapula, from an abnormal position to a normal or more normal position. The user first positions the shoulder engagement members 430 and 432 on top of the shoulders 414 and 416 and places the torso securing member 412 on the torso 420. The user then adjustably joins the rear torso engagement members 468 and 470 by attaching fastener 476 to flap 474. Next, the user adjustably joins the side flaps 444 and 446 of the front torso engagement member to the flaps 478 and 480 of the rear torso engagement member 428. The user then preferably positions and adjusts the tension of the various re-positioning members 422, 423 and 424. The user preferably positions the re-positioning member 423 in a downward direction across the portion of the rear torso engagement member containing the stiffening member, wrapping the end 423b around the opposite side of the subject 420a. The user then preferably positions the re-positioning members 422 and 424 to align the shoulders 414 and 416 near or adjacent to the ears of the subject 418 by pulling the shoulders 414 backwards in relation to the subject 418. Once installed on the subject 418, the user may rearwardly reposition shoulder 414 to increase proper alignment of the shoulder 414 by progressively attaching the shoulder engagement member fastener 534, the right rear torso engagement fastener 536 and the fastener 528 on the end 422b to the left rear torso engagement member 470. Likewise, the user may rearwardly reposition shoulder 416 by progressively attaching the shoulder engagement member fastener 538, the left rear torso engagement fastener 540 and the fastener 526 on the end 424b to the right rear torso engagement member 468. The fastener 540 preferably attaches to the re-positioning member 423 passing underneath re-positioning member 424.

The position adjustment device 410 of the present invention moves the wearer's shoulders 414 and 416 from an abnormally aligned position to a normal or more normal aligned position. The flexible and resilient material of the position adjustment device 410, permits a broad range of movement of abnormally positioned shoulders while re-positioning the shoulders. The position adjustment device 410 also assists in proper alignment of the scapula on the rib cage and improves shoulder girdle alignment. In certain situations, the position adjustment device 410 minimizes involuntary movement and enhances breathing by compressing the wearer's rib cage and supporting the diaphragm allowing the lungs to fill more completely. When the subject moves the shoulders, such as in the act of throwing a ball, the position adjustment device 410 helps keep the joints of the shoulders in their shoulder girdle (not shown) while enabling the subject to move the arms through a relatively broad range of motion. It is preferable that the position adjustment device 410 has the effects of decreasing the extent to which shoulders rotate forward, supporting the shoulder joint, aligning or aiding in the alignment of the humeral head to the glenoid fossa, aligning or aiding in the alignment of the scapula in relation to the rib cage, reducing or preventing pain, and playing a therapeutic role in the rehabilitation of both shoulder joints. This type of device provides wearers with dynamic bodily support during daily activity to improve and enhance their quality of life.

### IV. Fourth Embodiment

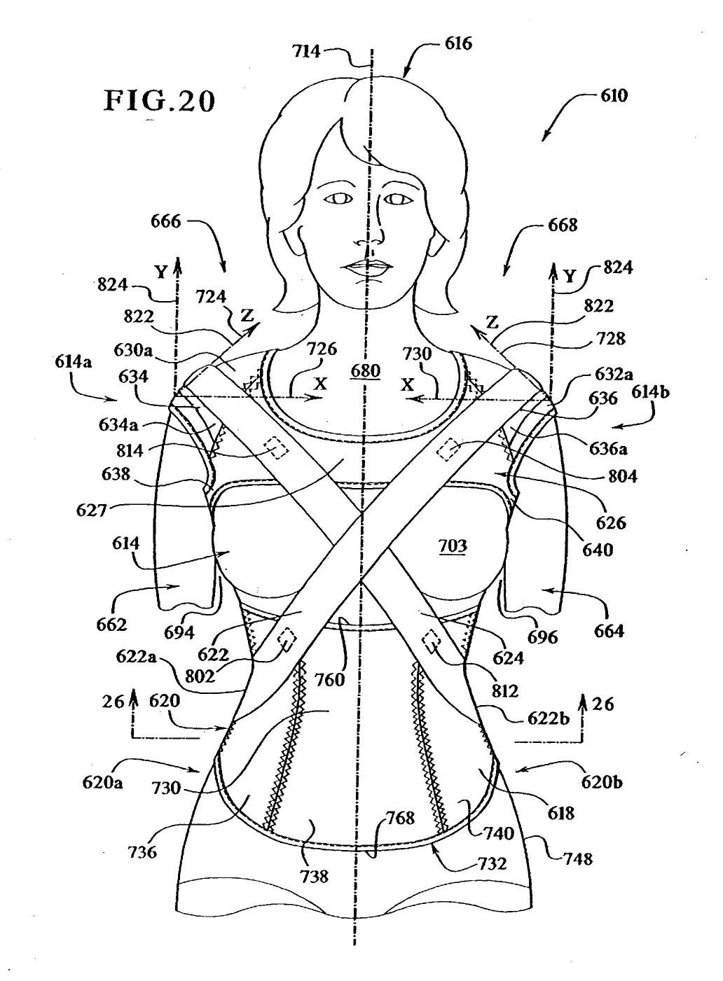

As illustrated in FIGS. 20 to 26, in one embodiment the position adjustment device 610 includes: (a) an upper torso securing member 612 which is engaged with the upper torso 614 or other upper body portions of the subject 616; (b) a lower torso securing member 618 which is engaged with the abdomen 620c and lower back 620d or lower torso 620 of the subject 616, which preferably serves as an anchor; and (c) a plurality of elongated re-positioning members 622 and 624 which connect the upper torso securing member 612 to the lower torso securing member 618.

### A. Upper Torso Securing Member

The upper torso securing member 612 includes: (a) a front (or anterior) torso engagement member 626; (b) a rear (or posterior) torso engagement member 628; (c) shoulder engagement members 630 and 632 which join the front torso engagement member 626 with the rear torso engagement member 628; and (d) elongated re-positioning members 634 and 636 which connect the front torso engagement member 626 with the rear torso engagement member 628.

The front torso engagement member 626 includes a cross-chest member 627 which extends across the upper chest and includes extension members or flaps 638 and 640 for connecting the cross-chest member 627 to the rear torso engagement member 628. The flaps 638 and 640 wrap rearwardly around both torso sides 614a and 614b to adjustably and removably attach to the rear torso engagement member 628. Specifically, re-adjustable fasteners 642 and 644, preferably of the hook-type, are attached to flaps 638 and 640, respectively, and engage the rear torso engagement member 628.

The rear torso engagement member 628 includes a right rear torso engagement member 646 and a left rear torso engagement member 648 which removably attach to the right rear torso engagement member 646. Specifically, the right rear torso engagement member 646 includes an extension member or flap 650 which removably connects to an extension member or flap 652 of the left rear torso engagement member 648 by a re-adjustable fastener 654. The fastener 654 is preferably of the hook type and is sewn or otherwise attached on the underside 656 of the right rear torso engagement member 646. The fastener 654 preferably extends the length of the flap 650 to thoroughly engage the flap 652 of the left rear torso engagement member 648. It should be appreciated that the fastener 654 can be attached to the underside of either the right or left rear torso engagement member 646 or 648.

The left rear torso engagement member 648 preferably includes a readjustable fastener 655 of the hook type sewn or otherwise attached on the underside 657 of the flap 652 to reversibly and adjustably attach the upper torso securing member 612 to the lower torso securing member 618.

The right rear torso engagement member 646 also includes a side extension flap 658 which extends around the side 614a of the upper torso 614 and under the right arm 662 of the subject 616 to thoroughly receive the re-adjustable fastener 642 on the flap 638 of the cross-chest member 627. Similarly, the left rear torso engagement member 648 includes a side extension flap 660 which thoroughly receives the fastener 644 of the flap 640 of the cross-chest member 627 under the left arm 664 of the subject 616. It should be appreciated that the fasteners 642 and 644 can also be attached to the underside of the flaps 650 and 652 of the rear torso engagement member 628 in order to attach to the flaps 638 and 640 of the cross-chest member 627.

The shoulder engagement members 630 and 632 wrap over the surface of the shoulders 666 and 668, respectively, and are attached by a plurality of stitches or otherwise in a non-removable fashion to the cross-chest member 627 and to each of the rear torso engagement members 646 and 648. The shoulder engagement member 630 is preferably divided into components 630a and 630b that allow the shoulder engaging member 630 to conform to shoulder 666. The components 630a and 630b are preferably joined to one another by a plurality of stitches or otherwise in a non-removable fashion. Similarly, the shoulder engagement member 632 includes components 632a and 632b which are preferably joined to one another by a plurality of stitches or otherwise in a non-removable fashion.

The cross-chest member 627 includes a neck wall 670 connected to: (a) a plurality of neck walls 672a and 672b of the shoulder engagement members 630a and 630b, respectively; (b) a plurality of neck walls 674a and 674b of the shoulder engagement members 636a and 636b, respectively; (c) a neck wall 676 of the right rear torso engagement member 646; and (d) and a neck wall 678 of the left rear torso engagement member 648. All of these neck walls 670, 672a, 672b, 674a, 674b, 676 and 678 define an opening 680 for the neck of the subject 616.

The cross-chest member 627 also includes an arm wall 682 of the flap 638 which is connected to a plurality of arm walls 684a and 684b of the shoulder engagement members 630a and 630b, respectively, and an arm wall 686 of the right rear torso engagement member flap 658 to define an opening 687 for the right arm 662 of the subject 616. Similarly, the cross-chest member 627 includes: (a) an arm wall 688 of the flap 640 which is connected to plurality of arm walls 690a and 690b of the shoulder engagement members 636a and 636b, respectively; and (b) an arm wall 692 of the left rear torso engagement member flap 660 to define an opening 693 for the left arm 664 of the subject 616.

In addition, the arm wall 682 of the flap 638 of the cross-chest member 627 in combination with the arm wall 686 of the right rear torso engagement member flap 650 defines an arm pit or under-arm cavity 694 of the right arm 662 of the subject 616. On the left side of subject 616, the arm wall 688 of the flap 640 of the cross-chest member 627 in combination with the arm wall 692 of the left rear torso engagement member 652 defines an arm pit or under-arm cavity 696 of the left arm 662 of the subject 616. The under-arm cavities 694 and 696 allow under-arm areas 662a and 664a of the subject 616 to receive ventilation. The under-arm cavities 694 and 696 also enhance the comfort of the subject 616 and facilitate the free movement of the arms 662 and 664 of the subject 616 when wearing the position adjustment device 610. The chest wall 698 of the cross-chest member 627 in combination with the torso walls 700 and 702 of the right and left rear torso engagement members 646 and 648 defines an opening 703 for the upper torso 614 including the chest, breasts or other portion of the upper torso 614 of the subject 616.

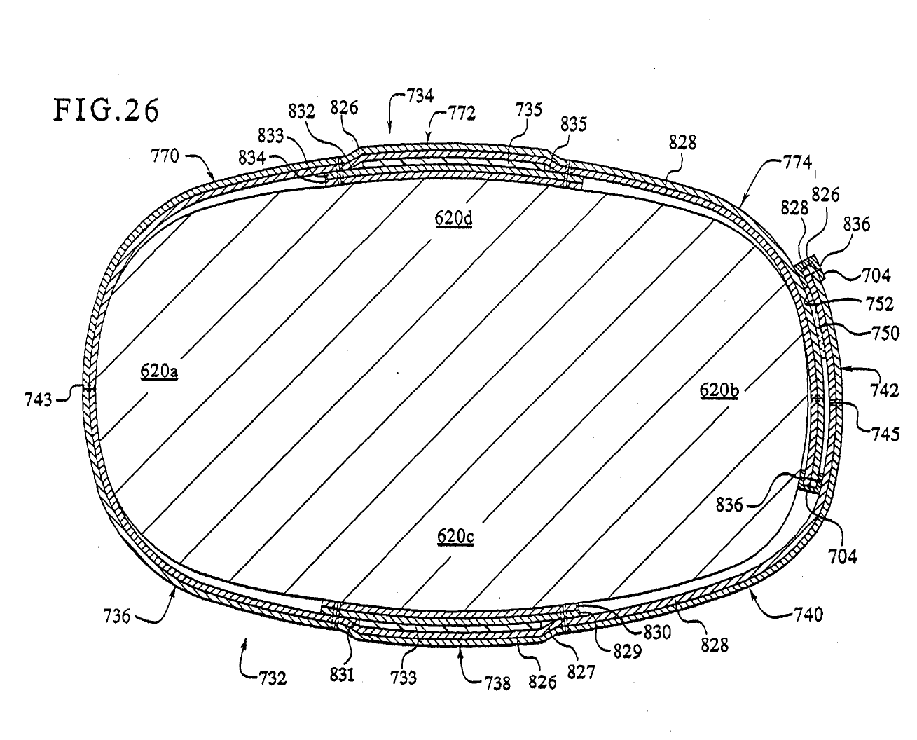

As best shown in FIG. 26, it is preferable that a reinforcement member 704 be attached to the edges of the torso securing member 612 to protect the layers of material of the position adjustment device 410 from separating, tearing, fraying or otherwise being damaged, and to aid in reinforcing and maintaining the shape of the brace. The reinforcement member 704 is preferably a binding or trim made of a flexible fabric or material having a relatively narrow uniform width sufficient to engage a portion of both an outer and inner layer 826 and 828, respectively, of the torso securing member 612. The reinforcement member 704 engages the surface of the outer layer 826 of the torso securing member 612 and is wrapped over the edges of the outer and inner layers 826 and 828 to engage the surface of the inner layer 828. The reinforcement member 704 is preferably non-removably secured to the surfaces of the outer and inner layers 826 and 828 by a plurality of stitches 836. The reinforcement member 704 preferably has a length sufficient to form a continuous edge along the contours of the torso securing member 612. It should be appreciated that other devices and techniques may be used to reinforce the attachment of the inner layer 828 to the outer layer 826 of the torso securing member 612.

In further description of the upper torso securing member 612, the re-positioning members 634 and 636 are preferably flexible members or straps. The re-positioning member 634 includes ends 634a and 634b, and the re-positioning member 636 includes ends 636a and 636b. The end 634a is preferably sewn or otherwise secured in a non-removable fashion to the seam 706 connecting the shoulder engagement member 630a to the cross-chest member 627. Similarly, the end 636a is preferably sewn or otherwise secured in a non-removable fashion to the seam 708 connecting the shoulder engagement member 636a to the cross-chest member 627. The ends 634a and 636a are positioned on the front torso engaging member 626 to allow re-positioning members 634 and 636 to engage a sufficient portion of the shoulders 666 and 668, respectively, to apply a relatively wide range of re-positioning force to the shoulders 666 and 668.

The re-positioning members 634 and 636 preferably wrap around the top of the shoulders 666 and 668 to engage the shoulders 666 and 668 and are directed horizontally or at any suitable angle 710 from a horizontal axis 712 across the rear torso engagement members 646 and 648. The re-positioning members 634 and 636 are preferably of a sufficient length to cross the vertical midline 714 of the back of subject 616. Ends 634b and 636b preferably include hook-type re-adjustable fasteners 716 and 718 on the undersides 720 and 722 of the re-positioning members 634 and 636, respectively. It is preferable that the re-adjustable fastener 716 attached to the end 634b of the re-positioning member 634 adjustably and removably attaches to the left rear torso engagement member 648. Similarly, it is preferable that the re-adjustable fastener 718 attached to the end 636b of the re-positioning member 636 adjustably and removably attaches to the right rear torso engagement member 646. It should be appreciated that elongated member 634 can be positioned over or underneath elongated member 636 at or near the midline 714 of the back of subject 616.

The re-positioning members 634 and 636 function as re-alignment or re-positioning members by applying a re-positioning force to both shoulders 666 and 668 of the subject 616. For the right shoulder 666, this re-positioning force includes: (a) a rearward or z-axis line of force 724 which pulls the shoulder 666 from the front of the subject 616 to the back of the subject 616; and (b) an inward or x-axis line of force 726 which compresses the shoulder 666 into the shoulder socket of the subject 616. For the left shoulder 668, this re-positioning force includes: (a) a rearward or z-axis line of force 728 which pulls the shoulder 668 from the front of the subject 616 to the back of the subject 616; and (b) an inward or x-axis line of force 730 which compresses the shoulder 668 into the shoulder socket of the subject 616. Accordingly, each of the re-positioning members 634 and 636 have a rearward line of force and an inward line of force. The re-positioning members 634 and 636 thereby dynamically re-position the shoulders 666 and 668 from an abnormal front position to a back position.

### C. Lower Torso Securing Member

The lower torso securing member 618 of the position adjustment device 612 preferably serves as an anchor for the securing member 612 and preferably includes: (a) a front (or anterior) torso engagement member 732; (b) a rear or backside (or posterior) torso engagement member 734; and (c) at least one stiffening member 733 and 735 connected to and preferably housed within the front torso engagement member 732 and the rear torso engagement member 734.

The front torso engagement member 732 preferably engages the abdomen 620c or lower back 620d or other lower body portions of the subject 616. The front torso engagement member 732 preferably includes: (a) a front right hip panel 736; (b) a front center abdominal panel 738; (c) a front left hip panel 740; and (d) a flap or extension member 742. The panels 736, 738 and 740, and flap 742 are preferably joined to one another by a plurality of stitches or otherwise in a non-removable fashion. The front right hip panel 736 of the front torso engagement member 732 is preferably sewn or otherwise secured in a non-removable fashion to the rear torso engagement member 734 of the lower torso securing member 618 at a seam 743. The front left hip panel 740 is preferably sewn or otherwise secured in a non-removable fashion to the flap or extension member 742 at a seam 745. The flap 742 extends around the hip 748 of the subject 616 where it overlaps and attaches to the rear torso engagement member 734 of the lower torso securing member 618. The flap 742 of the front torso engagement member 732 is adjustably and removably attached to the rear torso engagement member 734 by a re-adjustable fastener 750, preferably of a hook-type, that is non-removably attached on the underside 752 of the flap 742 of the front torso engagement member 732.

The front right hip panel 736, front center abdominal panel 738 and front left hip panel 740 have walls 754, 756, and 758, respectively, which form lower chest wall 760. The lower chest wall 760 further defines the abdominal space 620c in conjunction with the upper chest wall 698 of the upper torso securing member 612 that accommodates the chest or breast area 703 of the subject 616. The front right hip panel 736, front center abdominal panel 738, and front left hip panel 740 also have walls 762, 764, and 766, respectively, which form a pelvic wall 768. The pelvic wall 768 defines an opening which receives and engages the hips 748 of the subject 616.

The rear torso engagement member 734 preferably engages the lower back 620d or other body portions of the subject 616. The rear torso engagement member 734 preferably includes a plurality of panels: (a) a rear right hip panel 770, (b) a rear center back panel 772, and (c) a rear left hip panel 774. The panels are preferably joined to one another by a plurality of stitches or otherwise in a non-removable fashion. As described above, the rear right hip panel 770 of the rear torso engagement member 734 is preferably sewn or otherwise secured in a non-removable fashion to the front right hip panel 736 of the front torso engagement member 732 at the seam 743. The rear left panel 774 is preferably a flap or extension member that extends around the side 620b of the torso 620 where it engages the fastener 750 of the front torso engagement member of the securing member 732. It should be appreciated that the rear left panel 774 may have a flap to which the fastener 750 may be attached to engage the front left panel 740. The rear torso engagement member 734 of the lower torso securing member is preferably positioned to partially extend underneath the rear torso engagement member 628 of the upper torso securing member 612. However, the rear torso engagement member 734 of the lower torso securing member is preferably not attached to the rear torso engagement member 628 of the upper torso securing member 612.

The rear right hip panel 770, rear center back panel 772 and rear left hip panel 774 have walls 776, 778, and 780, respectively, which form a lower back wall 782. The lower back wall 782 further defines a back space 783 in conjunction with the lower torso walls 700 and 702 of the upper torso securing member 612. The back space 783 accommodates bending of the subject 616 and also provides ventilation to the subject's back. Similarly, the rear left hip panel 770, rear center abdominal panel 772 and rear left hip panel 774 have walls 784, 786, and 788, respectively, which form a pelvic wall 790. The pelvic wall connects to the pelvic wall 768 of the front torso engagement member 732 to receive and engage the hips 748.

Referring to FIGS. 20 and 26, the front center abdominal panel 738 preferably has an outer layer 826, an intermediate layer 827, an intermediate layer 829, and an inner layer 830. The stiffening member 733 is positioned between the intermediate layer 827 and the intermediate layer 829. The inner layer 830 is positioned to be placed against the skin of the subject 616. The stiffening member 733 preferably has a relatively flat shape and is positioned and sized to fill the space 831. The stiffening member 733 functions, in part, to stabilize and further support the abdomen 620c of the subject 616.

Similarly, the rear center back panel 772 preferably includes an outer layer 826, an intermediate layer 832, an intermediate layer 833, an inner layer 834 and a stiffening member 735 positioned between the intermediate layer 832 and the intermediate layer 833. The inner layer 834 is positioned to be placed against the skin of the subject 616. The stiffening member 735 preferably has a relatively flat shape and is positioned and sized to fill the space 835. The stiffening member in the rear torso engagement member 735 functions, in part, to stabilize and further support the lower back 620d of the subject 616.

It is preferable that a reinforcement member 850 be attached to the edges of torso securing member 618 to protect the layers of material of the position adjustment device 410 from separating, tearing, fraying or otherwise being damaged, and to aid in reinforcing and maintaining the shape of the brace. The reinforcement member 850 is preferably a binding or trim made of a flexible fabric or material having a relatively narrow uniform width sufficient to engage a portion of both outer and inner layers 826 and 828 of the torso securing member 618. The reinforcement member 850 engages the surface of the outer layer 826 of the torso securing member 618 and is wrapped over the edges of the outer and inner layers 826 and 828 to engage the surface of the inner layer 828. The reinforcement member 850 is preferably non-removably secured to the surfaces of the outer and inner layers 826 and 828 by a plurality of stitches 705. The reinforcement member 850 preferably has a length sufficient to form a continuous edge along the contours of the torso securing member 618. It should be appreciated that other devices and techniques may be used to reinforce the attachment of the inner layer 828 to the outer layer 826 of the torso securing member 618.

### D. Re-Positioning Members

The elongated re-positioning members 622 and 624 which can function as force redistribution members, are preferably resilient members or straps. The re-positioning member 622 includes ends 622a and 622b, and the re-positioning member 624 includes ends 624a and 624b. The end 622a is preferably sewn or otherwise secured to the seam 743 where the right front hip panel 736 and the right rear hip panel 770 are joined. Likewise, end 624a is preferably sewn or otherwise secured to the seam 745 where the left front hip panel 740 and the flap 742 are joined. The ends 622a and 624a are preferably oriented in the seams 744 and 746 such that the re-positioning members 622 and 624 can be directed toward each other to intersect at the approximate center of the front torso 614c. It should be appreciated that the re-positioning member 622 can be positioned over or underneath re-positioning member 624 at the approximate center of the torso 614c of subject 616. The re-positioning member 622 extends over the top of the shoulder 668. Likewise, the re-positioning member 624 extends over the top of the shoulder 666. The re-positioning members 622 and 624 are then directed down the back of the subject 616 toward the lower torso securing member 618. It is preferable that the re-positioning members 622 and 624 approach the midline 714 at an adjustable angle. However, it should be appreciated that the re-positioning members 622 and 624 may extend down the back parallel to the midline 714. The ends 622b and 624b adjustably and removably attach to the rear center panel 772 of the rear torso engagement member 734. The ends 622b and 624b preferably include hook-type re-adjustable fasteners 794 and 796 on the undersides 798 and 800 of the re-positioning members 622 and 624, respectively.

A plurality of re-adjustable fasteners, force distribution or fixing members 802 to 810, preferably of the hook-type, are spaced along the undersides 798 of the re-positioning member 622 to attach the re-positioning member 622 to portions of the position adjustment device 610. The fixing member 802 removably secures the re-positioning member 622 to the front torso engagement member 732 of the lower torso securing member 618. The fixing member 804 removably secures the re-positioning member 622 to the front torso engagement member 626 of the upper torso securing member 612. The fixing member 806 removably secures the re-positioning member 622 to the shoulder engagement member 636. The fixing member 808 removably secures the re-positioning member 622 to the rear torso engagement member 648 of the upper torso securing member 628. The fixing member 810 removably secures the re-positioning member 622 to the rear torso engagement member 734 of the lower torso securing member 618.

A plurality of readjustable fasteners, force distribution members or fixing members 812 to 820 are similarly positioned along the underside 800 of the repositioning member 624. The fixing members 812 to 820 are substantially horizontal to the fixing members 802 to 810 of the re-positioning member 622. The fixing member 812 removably secures the re-positioning member 624 to the front torso engagement member 732 of the lower torso securing member 618. The fixing member 814 removably secures the re-positioning member 624 to the front torso engagement member 626 of the upper torso securing member 612. The fixing member 816 removably secures the re-positioning member 624 to the shoulder engagement member 630. The fixing member 818 removably secures the re-positioning member 624 to the rear torso engagement member 646 of the upper torso securing member 628. The fixing member 820 removably secures the re-positioning members 624 to the rear torso engagement member 734 of the lower torso securing member 618.

The fixing members 802 to 820 re-distribute upper body forces along lines extending from the abdomen 620c to the lower back 620d, aiding in the proper body alignment and posture. Between each fixing member 802 to 820 are lines of force that distribute a re-positioning force over the length of the re-positioning members 622 and 624. This re-positioning force is applied to the torso 614 in relation to the lower body 620 and includes: (a) a rearward or z-axis line of force 822 which pulls the upper torso 614 from the front of the subject 616 to the back of the subject 616; and (b) a downward or y-axis line of force 824. Accordingly, the re-positioning members 622 and 624 have a rearward line of force 822 and a downward line of force 824. The re-positioning members 622 and 624 thereby dynamically re-position the torso 614 in relation to the lower body 620 in a rearward direction from an abnormal forward-leaning position to an upright or close to upright straight position, thereby improving the posture of the subject 616. In addition, the re-positioning members 622 and 624 function as springs or biasing members which apply a posture spring force. The posture spring force biases the upper torso backward relative to the lower back. This can provide both abdominal and lower back support while the wearer is sitting, standing, at rest or in motion.

### E. Materials

It is preferable that the securing members 612 and 618 are constructed of a soft and flexible fabric or material which provide additional re-positioning forces when the position adjustment device 610 is installed. The material of the securing members 612 and 618 preferably has two plies or layers 826 and 828, and, as described above with respect to the front and rear torso engagement members 732 and 734, two additional intermediate layers 829 and 830, and 833 and 834, respectively.

The outer layer 826 is preferably made of an unbroken nylon loop-type or UBL material that is relatively lightweight and is relatively resistant to moisture. The UBL, which preferably covers the entire outer layer 826, facilitates the installation and adjustment of the position adjustment device 610 of the present invention.

The material of the inner layer 828 as well as intermediate layers 830 and 834 are also relatively lightweight and preferably resistant to moisture. The inner layer 828 and the intermediate layers 830 and 834 preferably define a plurality of relatively uniformly distributed small openings or orifices. These orifices help expose the skin to air for ventilation purposes. It is also preferable that the inner layer 828 and the intermediate layers 830 and 834 have a hypoallergenic characteristic which enables the layers to frictionally interact with the skin to hold the position adjustment device 610 on the engaged body portion. One suitable material for the inner layer 828 and the intermediate layers 830 and 834 is a perforated neoprene material defining orifices having a size in the range of one and one-half to two millimeters, preferably two millimeters. It should be appreciated that various open-cell materials can be used as the material for the inner layer 828 and the intermediate layers 830 and 834.

In the example illustrated in FIGS. 20 to 26, the position adjustment device 610 is shown as having a front stiffening member and a rear stiffening member which preferably supports and stabilizes the abdomen and lower back, respectively of subject 616. It should be appreciated that, in other examples not illustrated, the adjustment device 610 can be adapted to include one or more suitable stiffening members in order to treat other body portions of the subject such as one or both scapulae. The stiffening members 733 and 735 housed within the front and rear torso engagement members 732 and 734, respectively, are preferably made of a plastic material or other material preferably resistant to moisture that has sufficient integrity to resist deformation.

The re-positioning members 634 and 636, and 622 and 624 are preferably constructed of a relatively soft, hook-compatible material on both sides of the straps. It is preferable that the re-positioning member 622 and 624 are constructed of resilient or elastic material, including, but not limited to, materials including one or more rubber, fibers or chords

The re-adjustable fasteners used in the position adjustment device 612 can include any suitable connectors which enable a plurality of parts to securely, yet removably, engage with one another. Such fasteners are preferably made of the hook-type material, although snaps, zippers and other suitable fasteners can be used. The hook portion of Velcro®, a commercially available fastener, is one suitable hook-type fastener which can be used in the position adjustment device 610.

The non-removable or permanent-type fasteners used in the position adjustment device 610 can includes any suitable fastener or fastening technique which securely and non-removably attaches one part to another part, including, without limitation, stitches, threads, adhesives, molding, pins and zippers.

It should also be appreciated that part or all of the position adjustment device 610 can also be constructed of a network of rubber cords, plastic hooks and/or cotton fabric.

### F. Installation

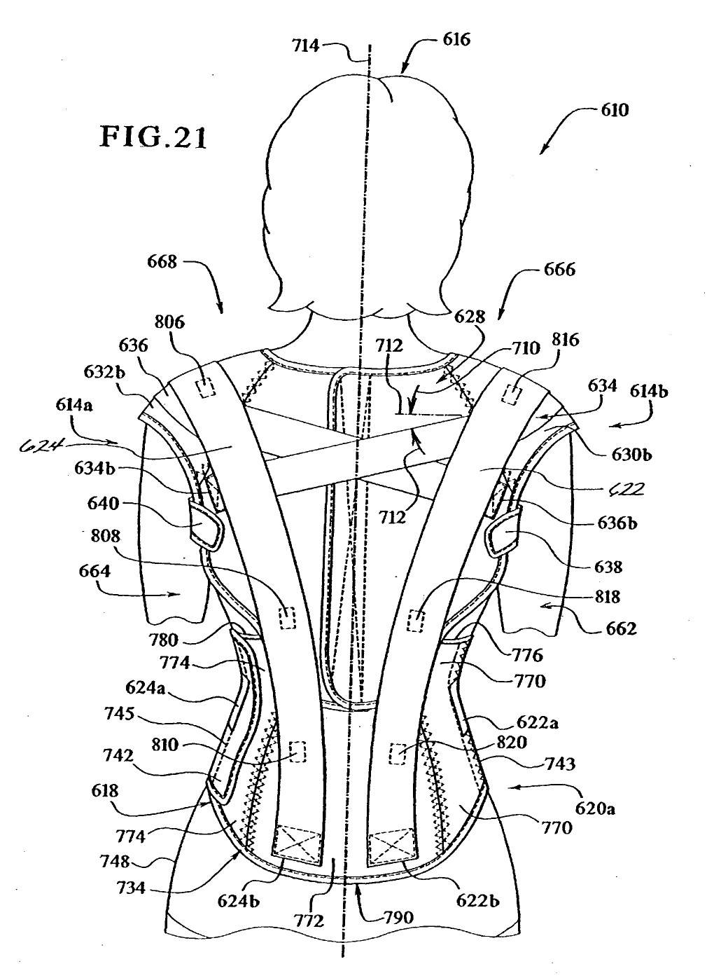

The position adjustment device 610 generally conforms to the configuration illustrated in FIGS. 20 and 21 and is adjustable by a user to re-position the shoulders and support the abdomen and lower back. The user first positions the shoulder engagement members 630 and 632 of the upper torso securing member 612 onto shoulders 666 and 668, respectively. Next, the user adjustably joins the respective readjustable fastener members 642, 644, 654, 716 and 718. The user attaches: (a) the fastener 642 of the cross-chest member flap 638 to the flap 658 of the rear torso engagement member 646; (b) the fastener 644 of the cross-chest member flap 640 to the flap 660 of the rear torso engagement member 648; and (c) the fastener 654 of the rear torso engagement member flap 650 to the flap 652 of the rear torso engagement member 648. Next, the user engages the shoulder 666 with re-positioning member 636 by wrapping around the front of the shoulder 666 and extending the re-positioning member 636 across the back 614d to attach to the rear torso engagement member 648. The user then engages the shoulder 668 with the re-positioning member 636 by wrapping around the front of the shoulder 668 and extending the re-positioning member 636 across the back 614d to attach to the rear torso engagement member 646.

After installing the upper torso securing member 612, the user can install the lower torso securing member 618. The user first positions the front center panel 738 on the abdomen 620c at the level of the hips 748 and wraps the rear torso engagement member 734 around the lower back 620d. The user then attaches the flap 742 of the front torso engagement member 732 to the back panel 774 by fastener 750. The upper torso securing member 612 is then attached to the rear center back panel 772 of the lower torso securing member 618 by the readjustable fastener 655 on the left rear torso engagement member 648. Upon securing the lower torso securing member 618, the user may then adjustably attach the re-positioning members 622 and 624. The user preferably begins by directing the re-positioning members 622 and 624 toward the center of the torso 614c, adjustably attaching the first pair of fixing members 802 and 812 of each re-positioning member 622 and 624, respectively, to the front torso engagement member 732. Next, the user crosses the re-positioning members 622 and 624 at the approximate center of the torso 614c and adjustably attaches the second pair of fixing members 804 and 814 to the cross-chest member 627 of the upper torso securing member 612. The user proceeds to direct the re-positioning member 622 over the shoulder 668 and the re-positioning member 624 over the shoulder 666. The user adjustably attaches the third pair of fixing members 806 and 816 to the shoulders 668 and 666, respectively. The fixing member 806 of the re-positioning member 622 is attached to the shoulder engagement member 636 and the fixing member 816 to the shoulder engagement member 630. The user then adjustably attaches the fourth pair of fixing members 808 and 818 to the rear torso engagement member 628. The user attaches fixing member 808 of the re-positioning member 622 to the left rear torso engagement member 648 and the fixing member 818 of the re-positioning member 624 to the right rear torso engagement member 646 of the upper torso securing member 618. The user then adjustably attaches the fixing member 810 of the re-positioning member 622 and the fixing member 820 of the re-positioning member 624 to the rear center panel 772 of the lower torso securing member 618. Ends 622b and 624b of the re-positioning members 622 and 624, respectively, are then attached to the rear center panel by the re-adjustable fasteners 794 and 796, respectively. It should be appreciated that the user may use any sequence of the steps listed above to install the position adjustment device 610.

The user preferably positions the re-positioning members 634 and 636 to align the shoulders 666 and 668 near or adjacent to the ears 808 by pulling the shoulders 662 and 664 backwards in relation to the subject 616. The tension of the biasing members or re-positioning members 622 and 624 can be adjusted incrementally by the series of fixing member pairs spaced along the length of the re-positioning members 622 and 624. The user operates the force distribution members 622 and 624 to provide alignment of the upper body with the lower body, thereby, improving the posture of the subject 616. In addition, when the subject 616 sits, stands, bends over or engages in other activity, the position adjustment device 610 positions the torso in relation to the lower body to dynamically support and improve the posture of the user at rest or through a range of activity.

It should be appreciated that, in other examples not illustrated, the position adjustment device 610 can be adapted to include a plurality of stiffening members which engage one or more abnormally positioned body portions or body portions in need of support or stabilization.

The position adjustment device 610, constructed of a flexible and relatively soft fabric or material, permits a broad range of movement of abnormally positioned shoulders while re-positioning the shoulders to normal or near normal positions. The position adjustment device 610 also allows the wearer to maintain appropriate posture through a broad range of movement. It is preferable that the position adjustment device 610 has the effects of supporting the shoulder joint, aligning or aiding in the alignment of the humeral head to the glenoid fossa, providing abdominal support and thoracic-lumbar stabilization, reducing or preventing pain, decreasing load on tissues, and playing a therapeutic role in the rehabilitation of the shoulder joint and lower back. This type of device provides wearers with dynamic bodily support during daily activity to improve and enhance their quality of life.

### V. Fifth Embodiment

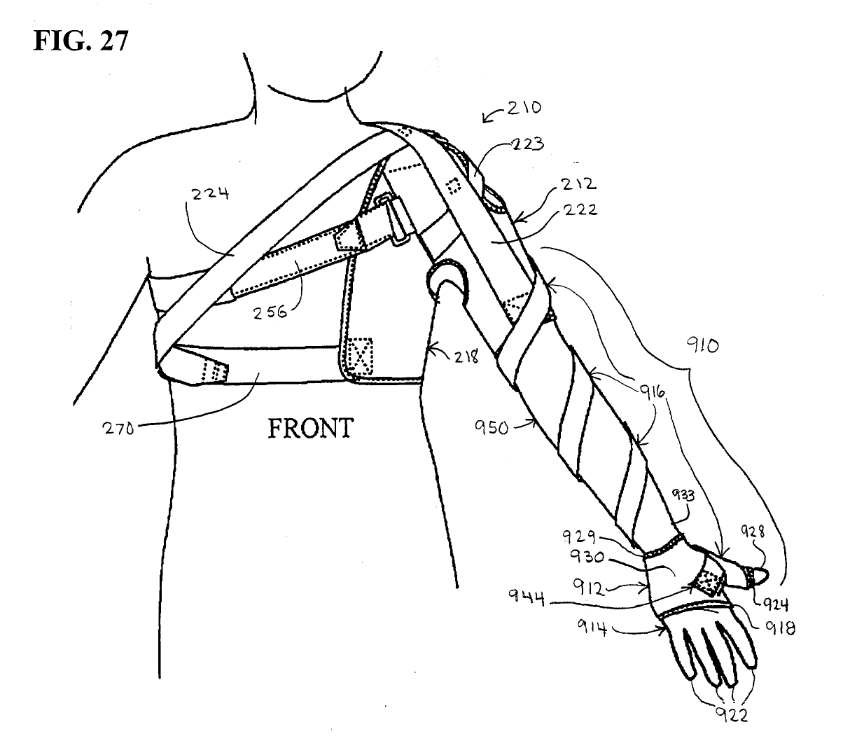

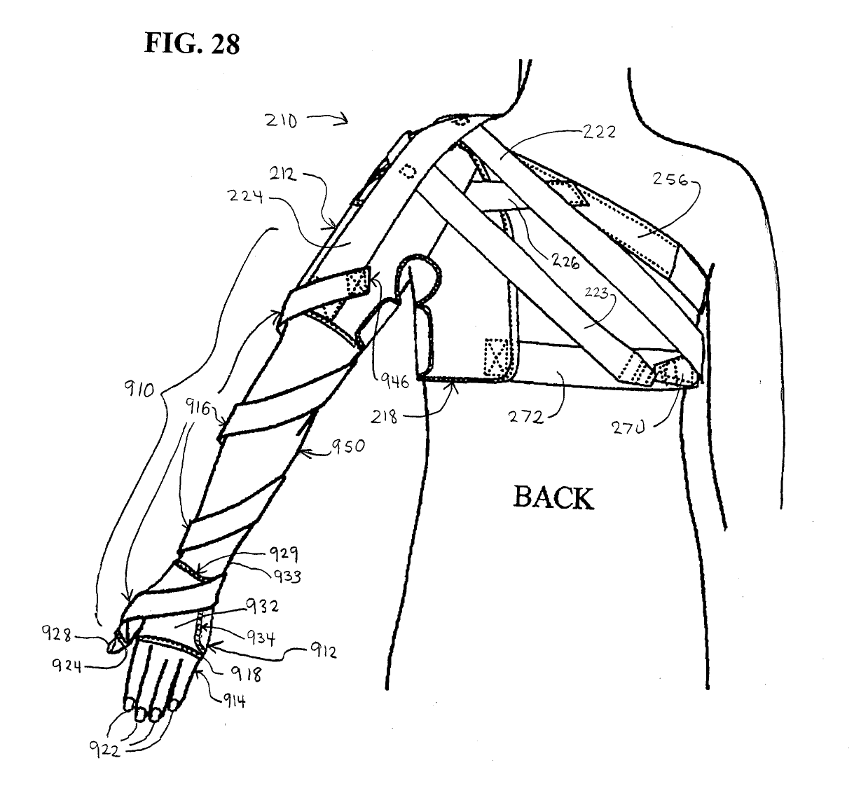

As illustrated in FIGS. 27 to 33, in one embodiment the present invention includes an extremity position adjustment device 910 which is adapted for use in conjunction with any suitable body portion securing member, including, without limitation, position device 10, position adjustment device 210, position adjustment device 410 and position adjustment device 610. One embodiment includes an assembly which includes any one of these securing members in combination with the extremity position adjustment device 910.

In the embodiment illustrated in FIGS. 27 to 33, the body portion securing member is the position adjustment device 210 described above which includes an additional elongated re-positioning member 223. Here, the extremity position adjustment device 910 includes: (a) an extremity securing member 912 which secures to an extremity such as a foot or hand, preferably a hand 914; and (b) an elongated extremity re-positioning member 916 which operatively couples the extremity securing member 912 to the position adjustment device 210.

### A. Extremity Securing Member

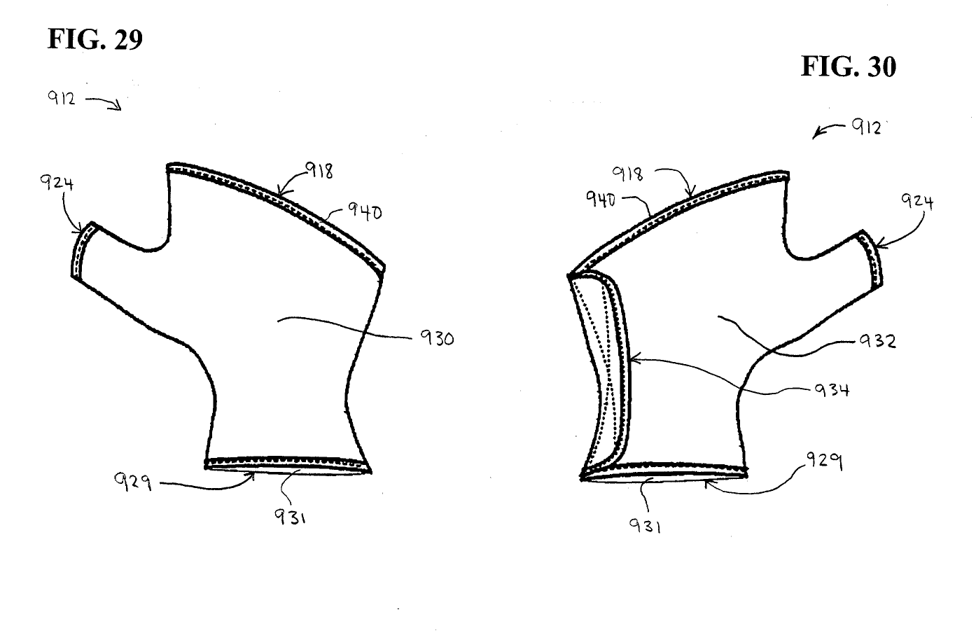

As best illustrated in FIGS. 29 through 32, the extremity securing member 912 includes: (a) at least one finger wall 918 which defines a finger opening 924 for receiving a plurality of fingers 922; (b) a thumb wall 924 which defines a thumb opening 926 for receiving a thumb 928; (c) a wrist wall 929 which defines a wrist opening 931 for receiving wrist 933; (d) a palm portion 930 which engages the palm of the hand; (e) a backhand portion 932 which engages the back of the hand; and (f) a plurality of ends 934 and 936 which are removably attachable to one another. The end 934 preferably includes a re-adjustable fastener 938 which enables the end 934 to removably attach to the end 936. In addition, the extremity re-positioning member 916 preferably includes a reinforcement member or reinforcement edge 940 secured to the perimeter of the extremity securing member 912.

### B. Extremity Re-Positioning Member

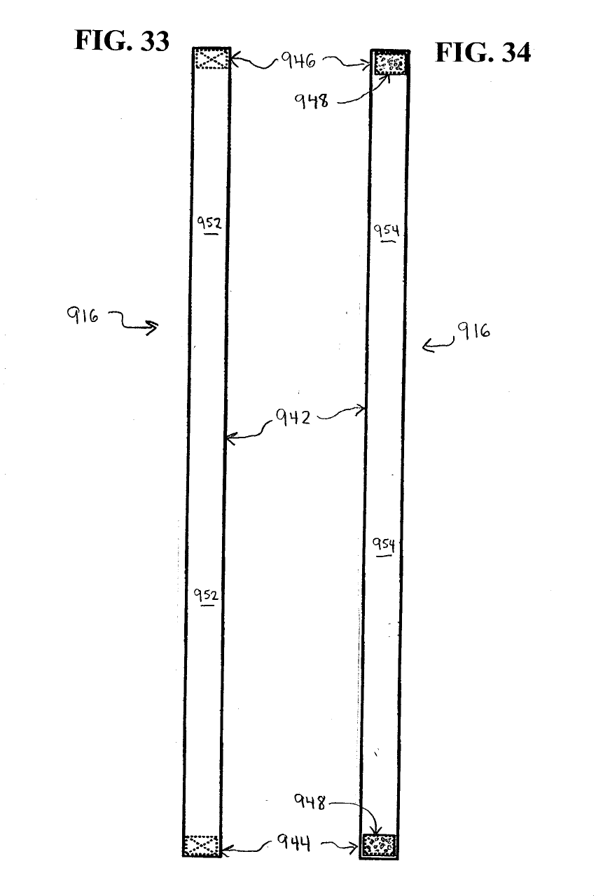

As best illustrated in FIGS. 33 and 34, the extremity re-positioning member 916 preferably includes an elongated portion 942 having an end 944 which is removably attachable to the palm portion 930 of the extremity securing member 912; and (b) an end 946 which is removably attachable to the arm securing member 212 of the position adjustment device 210. Each end 944 and 946 includes a re-adjustable fastener 948 which facilitates the removable attachment of the extremity re-positioning member 916 to the extremity securing member 912 and to the position adjustment device 210.

The extremity re-positioning member 916 is preferably a strap, which, when wound around the arm 950, applies a torsional or rotational force to the hand 914 as described below. In this capacity, the extremity re-positioning member 916 functions as a torsional or biasing member having torsional, biasing and/or rotational force characteristics.

### C. Materials

It is preferable that the extremity positioning device 910 is constructed of a relatively soft and flexible fabric or material. It is preferable that the material used to construct the extremity position adjustment device 910 has elastic or resilient characteristics which provide additional re-positioning forces when the extremity position adjustment device 910 is installed.

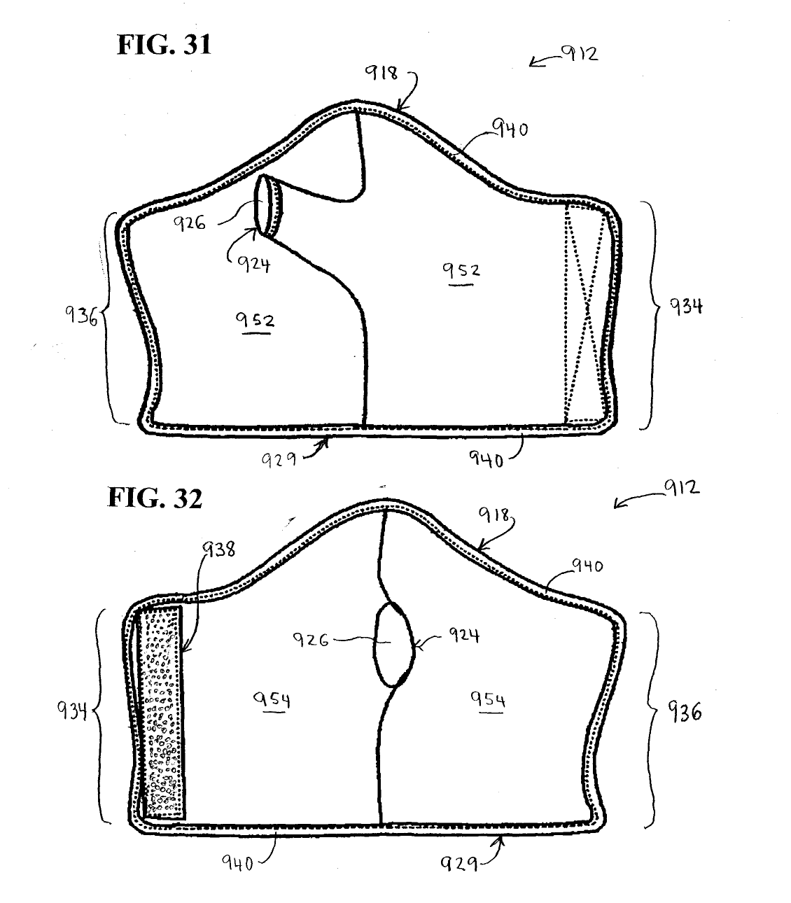

Referring to FIGS. 31 through 34, the material of the extremity position adjustment device 910 preferably has two plys or two layers defining an exterior surface 952 and an interior surface 954. The exterior surface 952 is preferably made of an unbroken nylon loop type or UBL material that is relatively light weight and is relatively resistant to moisture. The UBL, which preferably covers the entire exterior of the extremity adjustment device 910, facilitates the installation and adjustment of the extremity adjustment device 910 of the present invention.

The interior surface 954 preferably defines a plurality of relatively uniformly distributed small openings or orifices. These orifices help expose the skin to air for ventilation purposes. It is also preferable the interior surface 954 has a hypoallergenic characteristic which enables the interior surface 954 to frictionally interact with the skin to hold the position of the extremity adjustment position device 910 on the engaged body portion. For example, this friction increaser characteristic of the interior surface 954 of the re-positioning member 916 facilitates the distribution of force from the hand 914 to the position adjustment device 210. One suitable material for the interior surface 954 is a perforated neoprene material defining orifices having a size in the range of one and one half to two millimeters, preferably two millimeters. It should be appreciated that various open-cell materials can be used as the material for the interior surface 954.

The re-adjustable fasteners 938 and 948 used in the extremity position adjustment device 910 can include any suitable connectors which enable a plurality of parts to securely, yet removably, engage with one another. Such fasteners preferably are made of the hook-type material, although snaps, zippers, and other suitable fasteners can be used. Velcro®, a commercially available fastener, is one suitable hook-type fastener which can be used in the extremity position adjustment device 910.

The non-removable or permanent type fasteners used in the position adjustment device 910 can include any suitable fastener or fastening technique which securely and non-removably attaches one part to another part including, without limitation, stitches, threads, adhesives, molding, pins, and zippers.

### D. Installation

The extremity position adjustment device of the present invention can be installed on any right-sided extremity such as a right hand or a right foot or on any left-sided extremity such as a left hand or a left foot. In the examples illustrated, the extremity position adjustment device 910 is installed on the left hand 914 and left arm 950. In order to install the extremity position adjustment device 910, the user can insert his or her thumb 928 into the thumb opening 926, wrap the extremity securing member 912 around the hand 914 and attach the end 934 to the end 936. Alternately, the user can first attach the end 934 to the end 936 and then insert the hand 914 into the extremity securing member 912 so that the thumb 928 protrudes through the thumb wall 924 and the fingers 922 protrude through the finger opening 920.

Next, the user attaches the end 944 of the re-positioning member 916 at or near the center of the palm portion 930. With the palm of the hand 914 facing upward, the user wraps the re-positioning member 916 around the thumb 928 initially directing the loop from the palm side of the thumb to the back side of the thumb. After completing one loop around the thumb 928, the user directs the re-positioning member 916 along the back of the wrist and then winds or loops the re-positioning member 916 around the arm 950 at least once, but preferably a plurality of times. Then the user attaches the end 946 of the re-positioning member 916 to a desired location on the arm securing member 212, preferably near the tricep area. The user can adjust the degree of rotational force applied to the hand 914 by varying the position of the end 946 on the securing member 212.

The extremity position adjustment device 910 of the present invention can aid in the re-positioning of an extremity, such as a hand, from an abnormal position to a normal or more normal position. In one embodiment, the extremity position adjustment device 910 is useful in treating subjects whose shoulders are abnormally positioned which causes their hands to be abnormally pronated. In this case, the extremity position adjustment device 910 moves the hand to a suponated or more suponated position. It should be appreciated that the extremity position adjustment device of the present invention can be adapted to treat various medical conditions by applying a rotational or torsional force to an extremity in any desired direction, such as clockwise or counterclockwise. When the subject moves his or her arms, such as in the act of throwing a ball or writing a letter, the position adjustment device 910 helps keep the subject's hand in a normal or more normal position while enabling the subject to move his/her hands and arms through a relatively broad range of motion. This type of device provides wearers with dynamic extremity support during daily activity to improve and enhance their quality of life.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A device for use in conjunction with a body portion securing member for adjusting a position of an extremity of a subject, the device comprising:
an extremity securing member adapted to engage the extremity; and
at least one elongated member which has a first end connectable to the extremity securing member and a second end connectable to the body portion securing member.

2. The device of claim 1, wherein the extremity securing member has a first end and a second end, the first end removably attachable to the second end.

3. The device of claim 1, wherein a portion of the extremity securing member is removably attachable to the first end of the elongated member.

4. The device of claim 1, wherein the elongated member has a length so as to enable the elongated member to be wrapped around a limb of the subject a plurality of times while the first end is attached to the extremity securing member and while the second end is attached to the body portion securing member.

5. The device of claim 1, wherein a portion of the elongated member between the first end and the second end of the elongated securing member has a friction increaser characteristic.

6. The device of claim 1, wherein the body portion securing member includes a limb securing member.

7. The device of claim 1, wherein the extremity securing member includes a hand securing member.

8. The device of claim 1, wherein the body portion securing member includes an arm securing member.

9. The device of claim 1, wherein the body portion securing member includes a shoulder securing member.

10. An assembly for adjusting a position of a first body portion relative to a second body portion and for adjusting a position of an extremity, the assembly comprising:
a first securing member adapted to engage the first body portion;
a second securing member adapted to engage the second body portion, the second securing member operatively coupled to the first securing member;
an extremity securing member adapted to engage the extremity; and
at least one elongated member which has a first end attachable to the extremity securing member and a second end attachable to the first securing member.

11. The device of claim 10, wherein the extremity securing member has a first end and a second end, the first end removably attachable to the second end.

12. The device of claim 10, wherein a portion of the extremity securing member is removably attachable to the first end of the elongated member.

13. The device of claim 10, wherein the elongated member has a length so as to enable the elongated member to be wrapped around a limb of the subject a plurality of times while the first end is attached to the extremity securing member and while the second end is attached to the first securing member.

14. The device of claim 13, wherein the elongated member has a torsional force characteristic.

15. The device of claim 10, wherein the elongated member has a rotational biasing characteristic.

16. The device of claim 10, wherein a portion of the elongated member between the first end and the second end of the elongated member has a friction increaser characteristic.

17. The device of claim 10, wherein the extremity securing member includes a hand securing member.

18. The device of claim 10, wherein the first securing member includes an arm securing member.

19. The device of claim 10, wherein the first securing member includes a shoulder securing member.

20. A device for use in conjunction with an upper arm securing member operatively coupled to a portion of a torso of a subject for adjusting a position of a hand of the subject, the device comprising:
a hand securing member adapted to engage the hand, the hand securing member defining an opening for a plurality of fingers, an opening for a thumb, the hand securing member including a first end and a second end, the first end removably attachable to the second end; and
at least one elongated member which has a first end removably attachable to the extremity securing member and a second end removably attachable to the upper arm securing member, the elongated member having a length so as to enable the elongated member to wrap around an arm of the subject a plurality of times while said first end is attached to the extremity securing member and while said second end is attached to the upper arm securing member.

21. The device of claim 20, wherein the elongated member has a torsional force characteristic.

22. The device of claim 20, wherein the elongated member has a rotational biasing characteristic.
